(19) United States
(12) Patent Application Publication
(10) Pub. No.: US 2011/0229880 A1
(43) Pub. Date: Sep. 22, 2011

(54) GENE SILENCING

(75) Inventors: Matthew Wood, Oxford (GB); Chris Sibley, Oxford (GB); Yiqi Seow, Singapore (SG)

(73) Assignee: ISIS INNOVATION LIMITED, Summertown (GB)

(21) Appl. No.: 13/063,408

(22) PCT Filed: Sep. 10, 2009
(86) PCT No.: PCT/GB2009/002173

§ 371 Date: Jun. 7, 2011

(30) Foreign Application Priority Data
Sep. 12, 2008 (GB) 0816778.5

(51) Int. Cl. C12Q 1/68; C12N 5/071; C12N 15/85

(52) U.S. Cl. 435/6.1; 435/375; 435/455

(57) ABSTRACT

The invention provides a mirtron or a gene capable of expressing a mirtron for use in modifying the expression of a target gene in a mammalian cell to prevent or treat a disease, the sequence of the mirtron comprising: (i) a 5′ splice site; (ii) a 3′ splice site; (iii) a branch-point recognition sequence; (iv) a 3′ polypyrimidine tract greater than 15 nucleotides in length; and (v) an antisense sequence that is at least partially complementary to a sequence in the target gene.

### FIELD OF THE INVENTION

The present invention relates to methods and tools for modifying gene expression in mammalian cells.

### BACKGROUND OF THE INVENTION

RNA interference (RNAi) is a recently discovered post-transcriptional gene silencing phenomena in which short double-stranded RNA (dsRNA) of 21-23nts directs transcript degradation or translational repression of target messenger RNAs (mRNA). This powerful, sequence-specific mechanism for gene silencing is conserved through all eukaryotes and is known to have important roles in development, physiological maintenance and disease. Since its discovery in 1998, much research has focused on exploiting this natural system in the hope that harnessing RNAi may prove a novel therapeutic strategy for multiple diseases.

In the endogenous micro RNA (miRNA) pathway (FIG. 1), non-coding RNA with hairpin forming potential is transcribed as a primary-miRNA (pri-miRNA) transcript with characteristic stem-loop structure. The pri-miRNA is processed by the nuclease Drosha to yield a pre-miRNA. Nuclear export by Exportin-5 and further processing by the nuclease Dicer produces the mature miRNA. This is subsequently recruited by the RNA-induced silencing complex (RISC) which retains the anti-sense strand and destroys the sense/passenger strand. The anti-sense strand is used to scan mRNA transcripts for sites of sequence-specific complementarity with the degree of base-pairing determining whether the endonuclease Argonaute-2 directs transcript degradation or translational repression. Complete base-pairing directs mRNA cleavage whereas the predominantly encountered partial base-pairing, including nucleotides 2-9 that form the seed region that is dis-proportionally involved in target binding, directs translational repression.

The endogenous pathway plays a crucial role in translational control of mRNA transcripts to aid spatial and temporal control of gene expression, and it has un-surprisingly been found to be heavily involved in developmental processes. Furthermore disregulation of certain miRNAs has been implicated in disease states such as cancer and neurodegeneration. In addition to understanding the endogenous roles of miRNAs, three entry points into the RNAi pathway have been synthetically exploited to date at the level of:

i) Pri-miRNAs with miRNA mimics

ii) Pre-miRNAs through DNA-encoded short-hairpin RNAs (shRNAs)

iii) Mature miRNAs by double-stranded short-interfering RNAs (siRNAs).

### SUMMARY OF THE INVENTION

The current inventors investigated a different class of non-coding RNA molecules termed “mirtrons”. Mirtrons are short introns, that mimic pre-miRNAs with regards to length and hairpin potential, which are released in the splicing pathway as branched lariats. These lariats are characterised by 3′ ends that are looped back to join a branch-point nucleotide through 2′-5′-phosphodiester linkage. The action of lariat debranching enzyme relieves this lariat lasso structure such that a linear nucleotide sequence is released. Due to the hairpin potential of the mirtron sequences pre-miRNA mimics are formed which are capable of bypassing Drosha to join the RNAi pathway (FIG. 2). This novel entry into the RNAi pathway was demonstrated in D. Melanogaster (Ruby et al., 2007).

The inventors have developed a synthetic mirtron expression system that allows the testing of the functionality of potential mirtron sequences. The system enables the determination of whether a potential mirtron sequence (i) can be spliced and (ii) can achieve gene silencing or knock down. Using this system, the sequence requirements of mammalian mirtrons have been investigated. Establishing these sequence requirements allows the possibility of designing mirtrons capable of targeting and modifying the expression of any desired gene. The inventors have provided proof of this principle by demonstrating for the first time that it is possible to target and knock down an endogenous mammalian gene using a synthetic designed mirtron.

The invention provides:

A mirtron or a gene capable of expressing a mirtron for use in modifying the expression of a target gene in a mammalian cell to prevent or treat a disease, the sequence of the mirtron comprising:

(i) a 5′ splice site;

(ii) a 3′ splice site;

(iii) a branch-point recognition sequence;

(iv) a 3′ polypyrimidine tract greater than 15 nucleotides in length; and

(v) an antisense sequence that is at least partially complementary to a sequence in the target gene.

The invention also provides:

A vector comprising a mirtron or gene capable of expressing a mirtron as defined herein;

A vector comprising (i) a gene capable of expressing a mirtron and (ii) a reporter gene, wherein (i) and (ii) are under the control of the same promoter for use in monitoring the delivery and/or expression of the mirtron in the target mammalian tissue; and

A method of modifying gene expression in a mammalian cell, comprising delivering a mirtron, gene or vector as defined in any one of the preceding claims to a mammalian cell in vitro.

The current invention provides a number of advantages over existing RNAi strategies. First, entry into the RNAi pathway bypasses Drosha thus reducing constraints on the endogenous miRNA pathway. The predicted pre-miRNA mimics that are formed are also different in structure to canonical pre-miRNAs raising the possibility that an alternative export route to Exportin-5 could be used. Whereas pre-miRNAs traditionally have a 2nt overhang at the 3′ end making them an Exportin-5 substrate, the predicted mammalian mirtrons showed a trend towards lnt overhangs at both 5′ and 3′ ends of the stem-loop which are not traditional substrates for Exportin-5. Reports have also been made suggesting lariat debranching enzyme has a cytoplasmic localisation implying that spliced introns may exit the nucleus as branched lariats. Both Drosha and Exportin-5 are known bottlenecks in the endogenous pathway, which can be saturated synthetically leading to lethal consequences. By-passing both Drosha and Exportin-5 is therefore of considerable benefit.

Furthermore, since mirtrons are delivered as introns within longer transcripts, expression can be under the control of a PolII promoter. PolII promoters allow tighter spatial control of expression thus minimising off-target effects of RNAi whilst additionally producing transcripts at endogenous levels rather than the high-turnover ubiquitous PolIII promoters traditionally used for shRNAs and miRNA mimics

A mirtron of the invention can be used to reduce the expression of or knock down a target gene such as a mutant disease-causing gene. In one aspect of the invention, the knock-down of the target gene is directly coupled to the replacement of an RNAi-resistant version of the target gene. Such a strategy may be a viable alternative to allele-specific silencing for the treatment of dominantly inherited disorders as it eliminates the ambiguity of how much knock-down and subsequent discrimination is required between wild-type and mutant alleles for therapeutic success. Furthermore it avoids necessary constraints of targeting the nucleotide sequence surrounding the mutation which may inherently not be suitable for RNAi. Finally it would allow the use of a PolII promoter for transcription of the pre-mRNA transcript containing both mirtron and RNAi resistant gene such that cell-specific expression may be achieved, unlike when using the ubiquitous PolIII promoters required for shRNA expression.

### BRIEF DESCRIPTION OF THE FIGURES

FIG. 1 illustrates the RNAi pathway (Hammond 2005).

FIG. 2 illustrates the mirtron pathway (Ruby et al., 2007).

FIG. 3 details the development of a synthetic mirtron delivery vector and demonstration of RNAi by the predicted mirtron, miR-877. A) eGFP was engineered to allow synthetic introns to be introduced. The synthetic intron was place between the eGFP exons 1 and 2. eGFP expression was under the control of the CMV promoter. B) The miR-877 target with completely homologous base-pairing, was fused to the Renilla luciferase gene in a dual luciferase encoding plasmid. C) miR-877 was inserted as an intron in eGFP. Additional constructs incorporated mutations made to the branch point and 5′ and 3′ splice sites of the intron. A similar length hairpin deficient NAD-intron was used as a control. Key: BP=Branch point mutation; US=Un-spliceable mutant; DUS=Double un-spliceable mutant; Scram=Scrambled mutant. D) RT-PCR demonstrates splicing efficiency of each construct. Unsuccessful splicing leads to a long, non-functional, eGFP transcript and splicing of the intron allows reconstitution of the two eGFP exons into a shortened functional eGFP transcript E) eGFP fluorescence demonstrates splicing efficiency of each construct. Expression of constructs demonstrated eGFP fluorescence with both the NAD and miR-877 introns in both its wild-type and scrambled forms. No eGFP was seen with the miR-877 variations with mutations made to splicing regulatory sequences. F) Dual luciferase screening revealed silencing of the miR-877 target with the miR-877 sequence alone when expressed as an intron within eGFP. Variants of each construct expressed as shRNAs off an independent promoter demonstrate that the mutations made which inhibit splicing do not alter the ability of the sequence to act as an RNAi effecter. Results are normalised ratios of renilla luciferase to firefly luciferase +/−S.D.

FIG. 4 shows a synthetic mirtron demonstrating RNAi against cyclophilin-B. A) A control siRNA sequence targeting human cyclophilin-B was incorporated into the backbone of the predicted mirtron, miR-1226 (miR-21C). B) eGFP fluorescence demonstrates splicing efficiency of each construct. C) Western blot analysis reveals knockdown of the cyclophilin-B target relative to a control NAD-intron (D) Dual Luciferase Assay. HEK cells were co-transfected with a psiCheck2.2 vector with the cyclophilin mirtron target site in the 3′ UTR of luciferase and Mirt21C construct or NADintron construct. The results (relative luminescence) are derived from three replicates and clearly show significant knockdown by Mirt21C compared to the NADintron control. (E) qRT-PCR results (relative levels of cyclophilin B mRNA) of three biological replicates for cyclophilin B shows significant knockdown by Mirt21C compared with NADintron control. The results are normalised to levels of GAPDH. Results are +/−S.D.

FIG. 5 shows a synthetic mirtron demonstrating RNAi against the Parkinson's Disease linked alpha-synuclein gene. A) eGFP fluorescence demonstrates splicing ability of pMirt A-syn 5 whilst dual luciferase screening using a synthetic target to this mirtron demonstrates a ˜25% silencing effect. B) Expression of this mirtron alongside a full length target transcript with fluorescent mCherry tag demonstrates a ˜25% reduction in target expression relative to controls.

FIG. 6 demonstrates the generation of a random library of synthetic mirtron sequences targeting the cyclophillin B gene. A) Following generation of a random library of synthetic mirtrons targeting the Cyclophillin B gene, GFP quantification reveals variation in the amount of splicing achieved with each construct. B) Dual luciferase screening of successfully spliced mirtron variants demonstrates variation in the amount of silencing achievable with randomly evolved mirtrons.

FIG. 7 illustrates the design of synthetic mirtrons using an algorithm designed in-house based on the rulesdescribed herein. It provides A) the results of the mirtron finder algorithm and B) the resultant mirtron designs with the antisense strand in the 5′ arm or the 3′ arm.

FIG. 8 shows effective knockdown of DMPK with a synthetic mirtron targeted against DMPK. A) Knockdown of a luciferase target by and fluorescence reading of different mirtrons against DMPK in a pEGFP-Mirt vector. B) Representative fluorescent images of respective mirtron vectors co-transfected with a luciferase target vector. C) Schematic of DMPK Mirt 5. D) Fluorescence reading of and knockdown of a luciferase target by DMPK Mirt 5, DMPK 5 shRNA, and DMPK Mirt 5 US with a mutation in the terminal guanosine of the 5′ splice site.

FIG. 9 shows knockdown of other targets by DMPK Mirt 5. A) Schematic of a codon-replaced RNAi-resistant target of DMPK Mirt 5 and the miR-877 target B) Knockdown of luciferase constructs incorporating RNAi-resistant target and miR-877 target by DMPK Mirt 5.

FIG. 10 demonstrates the combinatorial mirtron strategy. A) Schematic of the dual intron vector compared to pEGFP-Mirt-877 and pEGFP-Mirt-1226; B) knockdown of miR-877 target and miR1226 target by the respective mirtrons and the dual intron vector; C) fluorescent images of the dual intron vector as compared to the single intron vectors and NADPH intron.

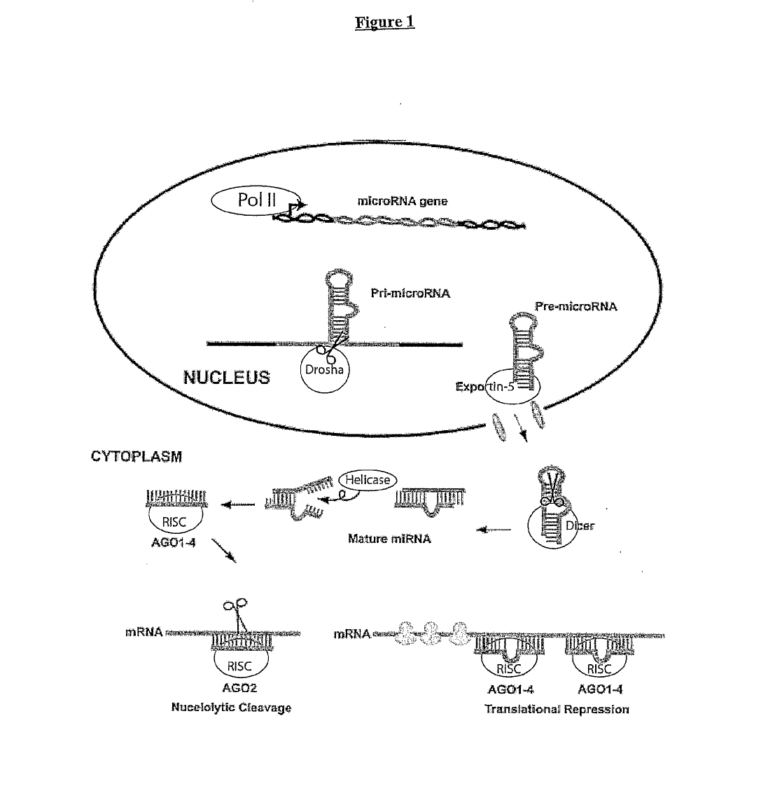

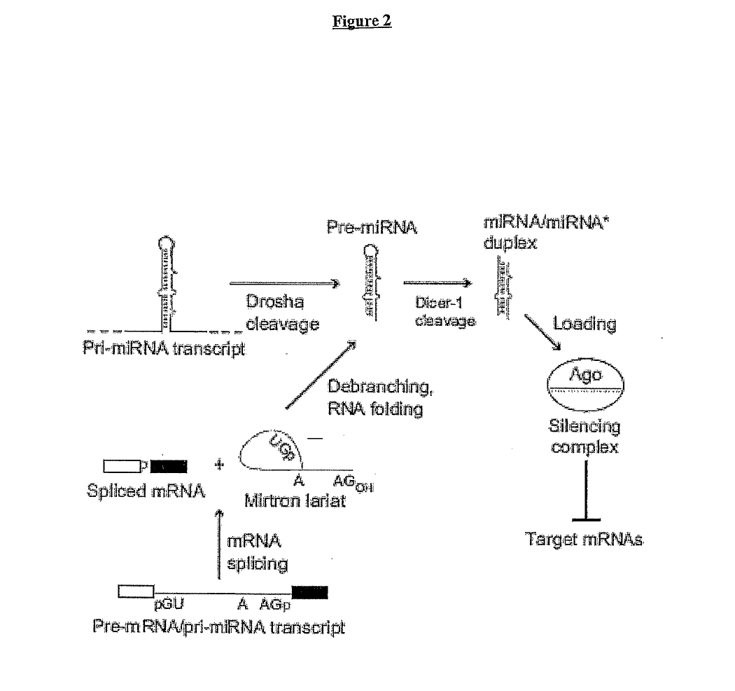

### DESCRIPTION OF SEQUENCES

SEQ ID NO: 1 to 64 are predicted mammalian mirtron sequences.

SEQ ID NO: 65 to 68 are forward and reverse primers used for generation of eGFP splicing reporter construct.

SEQ ID NO: 69 to 99 are mirtron insert sequences that were tested.

### DETAILED DESCRIPTION OF THE INVENTION

The current invention results from detailed investigations into, and validation of, a mammalian mirtron pathway. The current inventors have developed a synthetic mirtron expression system and have proved for the first time that putative mammalian mirtrons are functional and are dependent on splicing for entry into the RNAi pathway. The inventors have proved that it is possible to target and down-regulate any endogenous gene of interest using synthetic designed mirtrons, thereby establishing their therapeutic utility.

Example 1 details the construction of a synthetic mirtron expression system. In such a system, the mirtron sequence is placed between two exons of a reporter gene. Successful splicing results in the production of a shortened, functional mRNA transcript. An example of a suitable reporter gene that can be used is enhanced green fluorescent protein (eGFP). EGFP fluorescence indicates successful splicing.

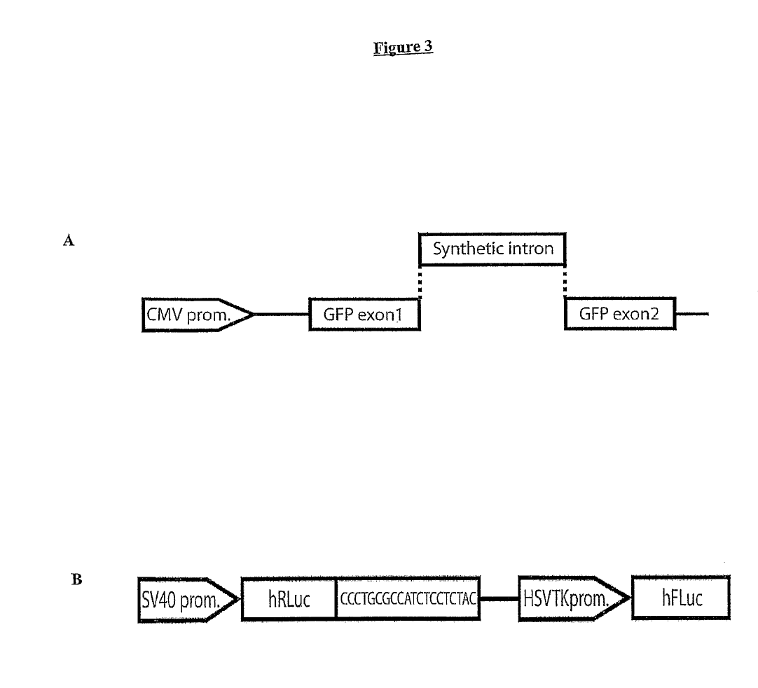

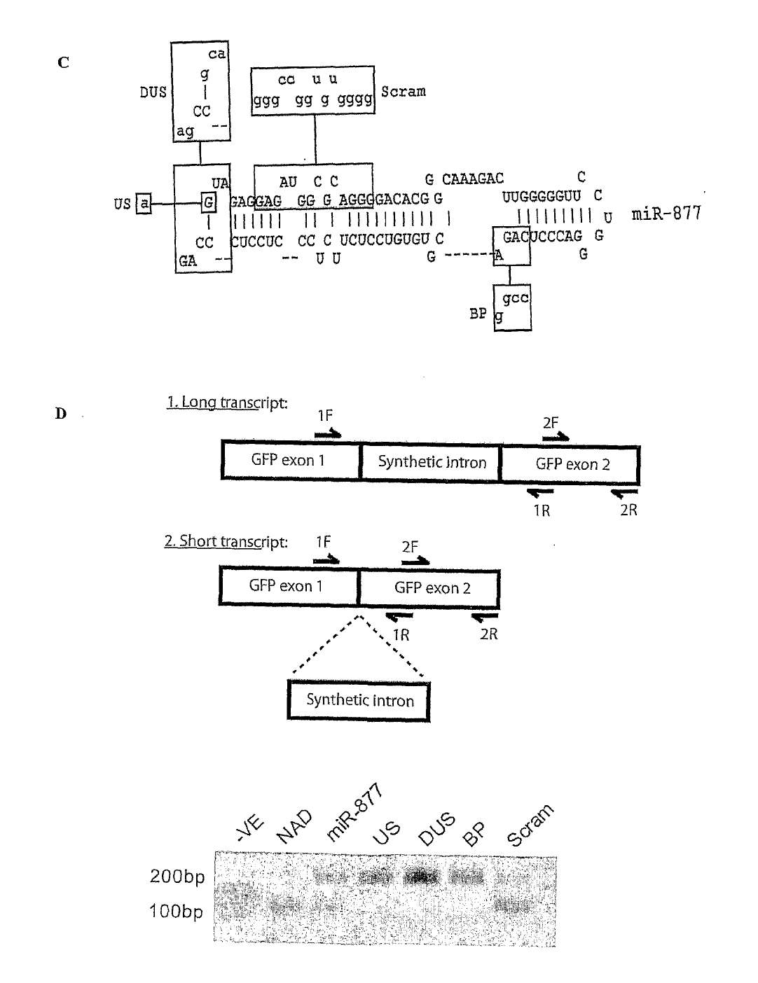

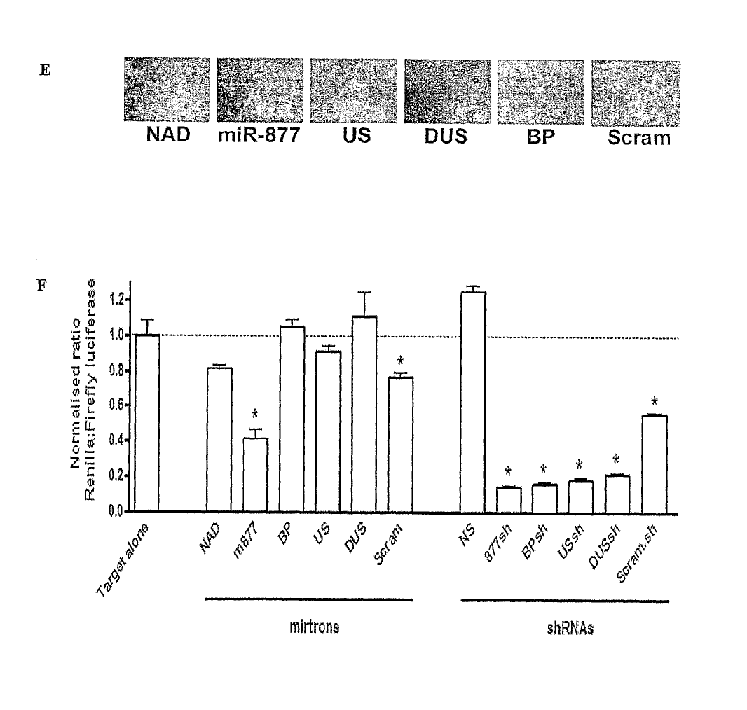

To test the ability of putative mammalian mirtrons to knock down its putative target in mammalian cells, the exact predicted mammalian mirtron sequence of miR-877 (Berezikov et al. 2007) was replicated as an intron into an eGFP transcript. Splicing dependence of knockdown was confirmed through the regeneration of the eGFP functional sequence, as witnessed by fluorescence and reverse-transcription PCR, and by the deficiency of this in constructs with mutated nucleotides located at key regions required for splicing (Example 1 and FIG. 3).

The model system can be used to test whether mammalian mirtrons possess the necessary traits that would be desirable for therapeutic purposes. These traits include the by-passing of Drosha and Exportin-5 in the RNAi pathway, the precise generation of pre-miRNA species, close coupling between miRNA species and ‘carrier’ mRNA transcripts and the use of Pol II promoters for transcription.

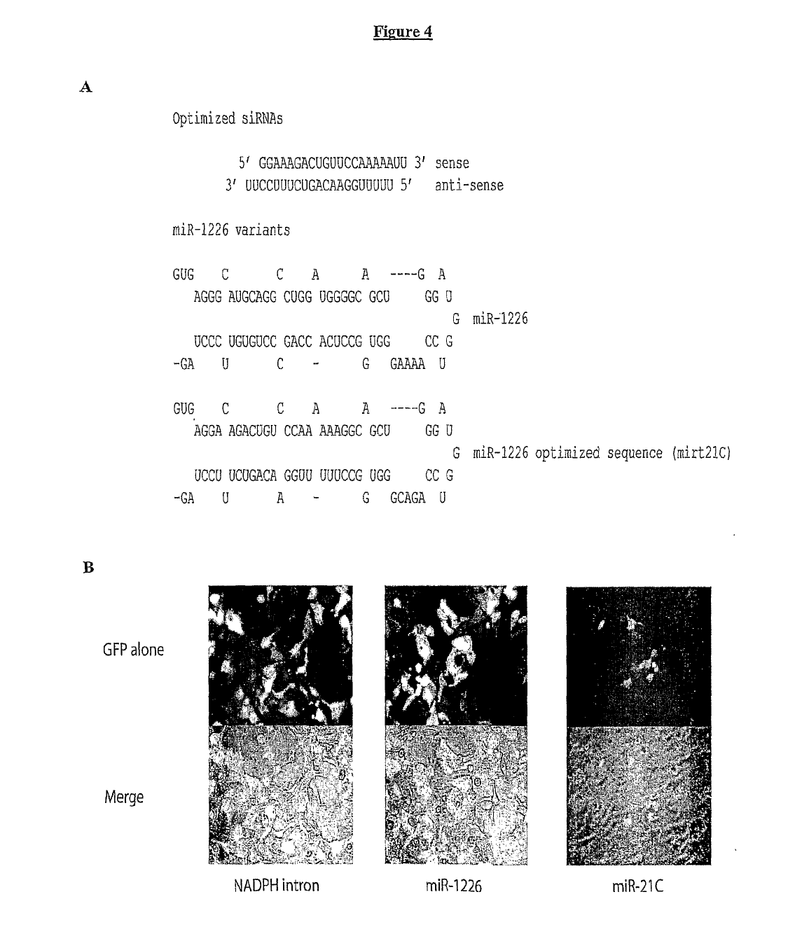

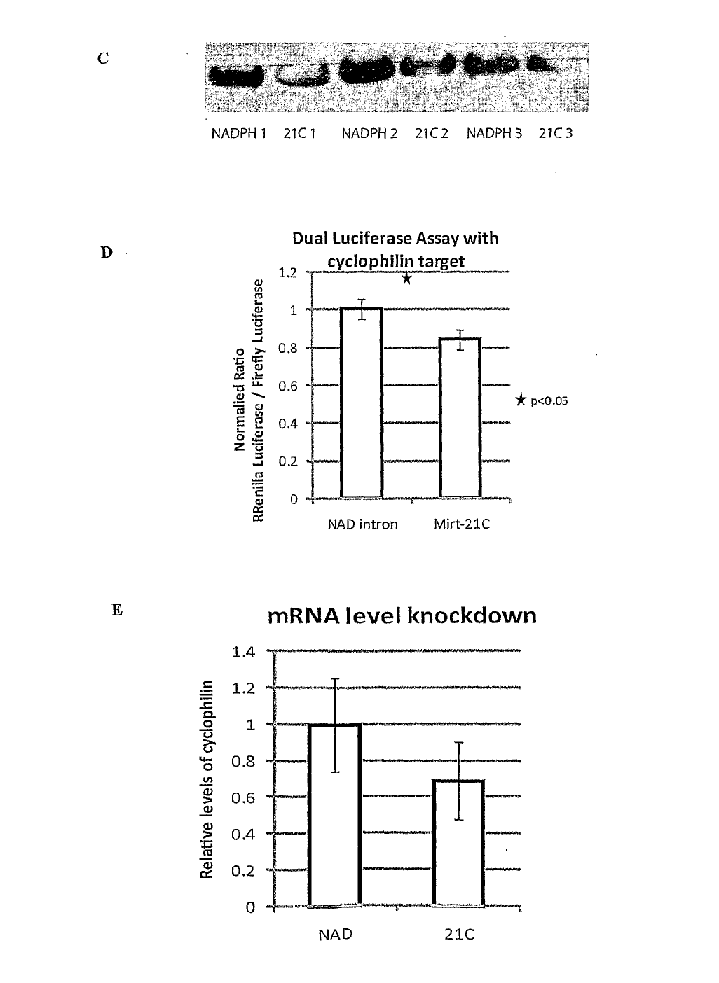

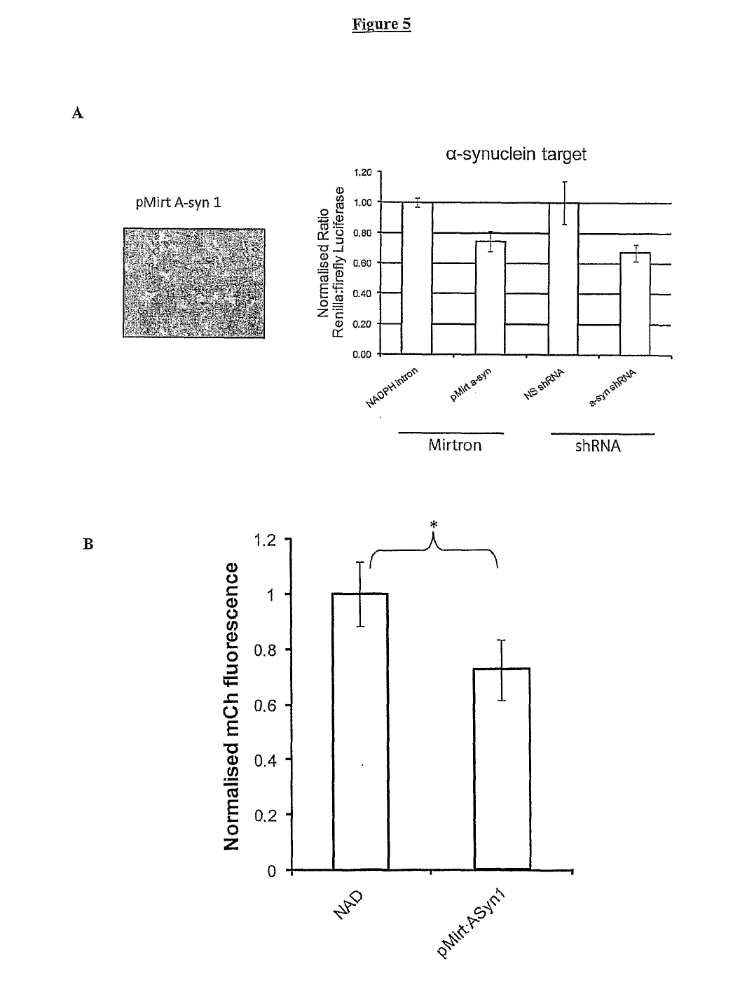

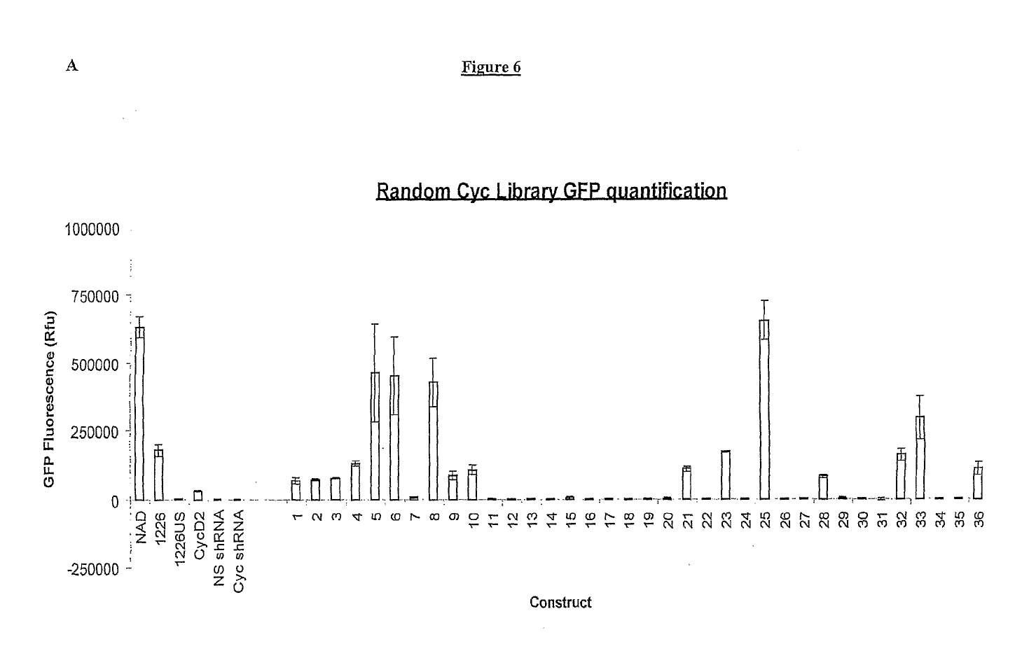

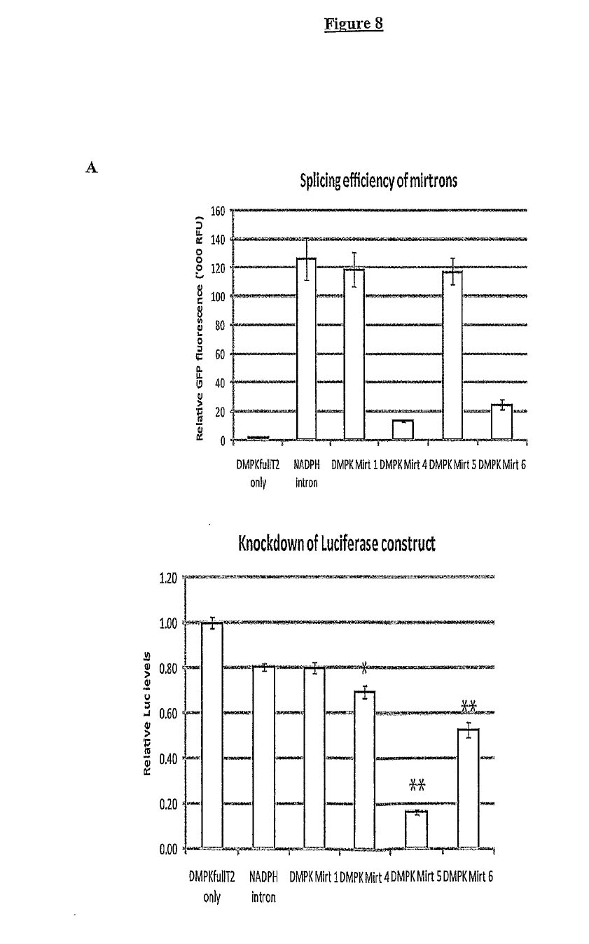

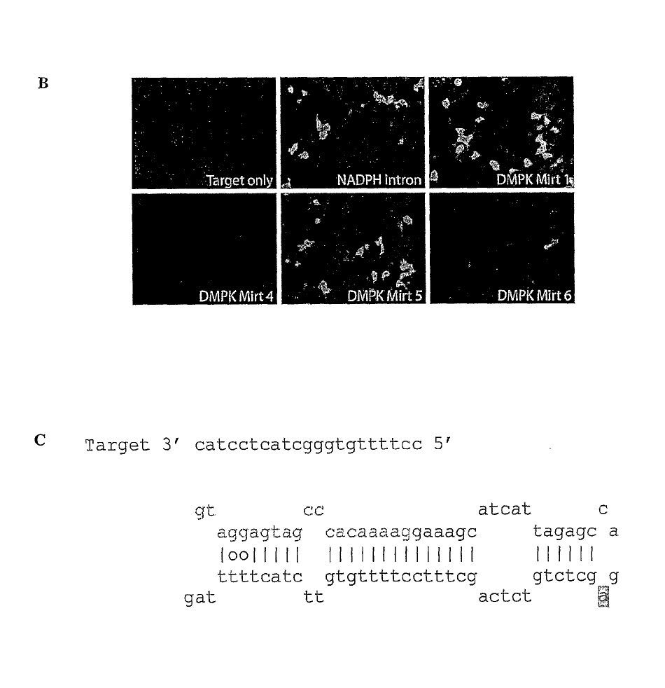

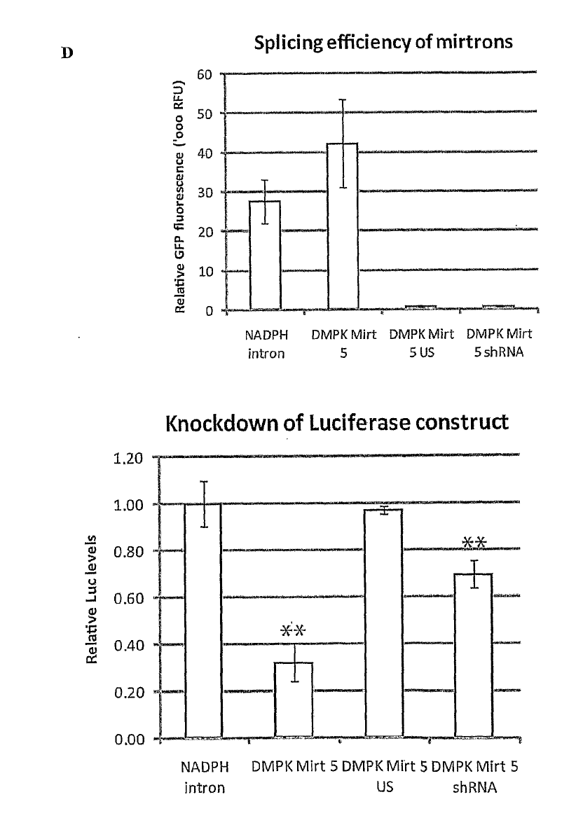

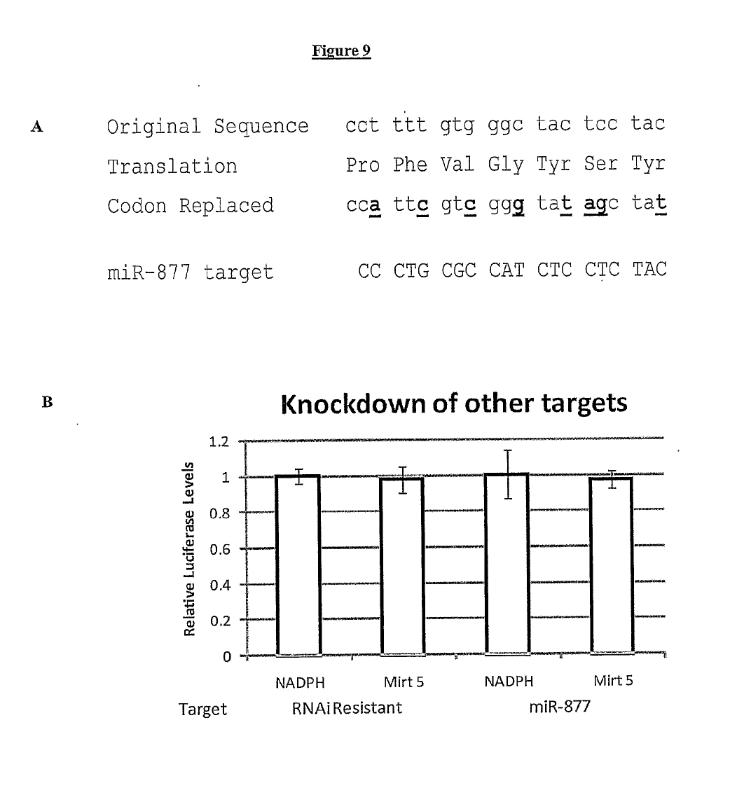

The system has been exploited to generate the first synthetic mirtron capable of silencing an endogenous gene of interest. Following several rounds of synthetic mirtron generation, a design utilising the miR-1226 backbone (Berezikov et al. 2007) with miR-877 optimised branchpoint and incorporating a siRNA sequence directed against cyclophilin-B, miR-21C, has demonstrated significant knockdown of target protein (Example 1 and FIG. 4). Additional mirtrons targeting a-synuclein (Example 1 and FIG. 5) and DMPK (Example 4 and FIG. 8) have since been generated, thus validating the use of synthetic mirtrons to knock down endogenous genes of interest.

Additionally, the rounds of mirtron generation leading up to this construct provide the first basis of rules for synthetic mirtron design with regards to what can and cannot be tolerated. Features of importance include the 5′ and 3′ splice-site consensus sequences of GU and AG respectively, the 3′ poly-pyrimidine tract leading into the 3′ splice site which must be of greater than 15nts in length and the presence of a branch-point recognition sequence.

The invention is directed to a mirtron or a gene capable of expressing a mirtron for use in modifying the expression of a target gene in a mammalian cell, preferably for use in preventing or treating a disease, the sequence of the mirtron comprising:

(i) a 5′ splice site;

(ii) a 3′ splice site;

(iii) a branch-point recognition sequence;

(iv) a 3′ polypyrimidine tract greater than 15 nucleotides in length; and

(v) an antisense sequence that is at least partially complementary to a sequence in the target gene.

The mirtrons of the current invention can be used for modifying the expression of a target gene in a mammalian cell. The mirtron can be used to reduce the expression of or cause silencing or knockdown of a target gene. The mirtrons of the invention cause reduction of expression of protein from the target gene. Typically, the reduction in amount of protein expressed in the cell may be at least 50% of the amount of protein expressed in the absence of the mirtron. Preferably, the reduction in expression is at least 55%, 60%, 65%, 70%, 75% or 80% or more preferably, at least 85%, 90% or 95%. A method for determining the relative amount of protein expressed may be any suitable method known in the art, for example Western blotting.

The mirtrons of the invention offer significant advantages over other traditional miRNA expression systems:

Mirtrons bypass Drosha and potentially also Exportin-5 in the RNAi pathway. These are bottlenecks in the pathway that limit other miRNA expression systems. Saturation of the RNAi pathway at these points can lead to interference of endogenous miRNA processing and result in toxic effects.

The use of site-specific RNA polymerase II promoters driving transgene mRNA, and hence mirtron, expression, can minimise potential off-target effects encountered when exploiting the RNAi pathway.

Gene knockdown can be coupled to a gene replacement that is knockdown-insensitive (codon-replaced), with the mirtron placed as an intron within the knockdown-insensitive gene being delivered. If placed under the endogenous promoter the miRNA expression is tightly coupled to the target gene expression without compromising on replacement gene expression, reducing the risk of off-target effects.

Allows easy establishment of therapeutic dosages of miRNA required for phenotypic recovery much more easily using a reporter gene system, such as eGFP, as reporter gene expression is tightly coupled to miRNA expression.

Allows sustained expression of miRNA without frequent repeated administration as is the limit for siRNA.

Allows the production of homogenous population of miRNA with clearly defined ends, minimizing potential off-target effects unlike pre-miRNAs and shRNAs.

Mirtron Design

The current inventors went through a rigorous design process to establish rules required for functional mammalian mirtrons. These efforts enable the design of mirtrons to effect silencing or knock down of any mammalian gene of interest. The inventors developed a system to test the functionality of each mirtron during the iterative design process.

An eGFP plasmid was engineered to allow any synthetic intron to be inserted in order to test splicing efficiency (Example 1). Successful splicing results in eGFP fluorescence due to the production of a shortened, functional mRNA transcript for eGFP. Splicing can also be tested by RT-PCR. Any suitable splicing reporter system could be used to test splicing of a predicted mirtron.

To test the ability of the designed mirtrons to work in mammalian cells, the target sequence of the test mirtron was fused to the renilla luciferase gene in a dual-luciferase cassette (FIG. 3B). By measuring the amount of renilla luciferase activity compared to the control firefly luciferase activity it is possible to determine the silencing capability of the test mirtron.

Using this system, the inventors have established rules and requirements for functionality of synthetic mirtrons. The sequence of the mirtron according to the invention comprises:

(i) a 5′ splice site;

(ii) a 3′ splice site;

(iii) a branch-point recognition sequence;

(iv) a 3′ polypyrimidine tract greater than 15 nucleotides in length;

(v) an antisense sequence that is at least partially complementary to a sequence in the target gene.

These requirements for a functional mirtron are now discussed in more detail.

Splicing Ability

The first requirement of the mirtron is the ability to be spliced out. This involves a 5′ splice site that can be recognized by U1 or U11 snRNA, a branch point that can be recognized by U2 or U12 snRNA, a 3′ splice site consensus sequence and a polypyrimidine tract between the branch point and the 3′ splice site on the 3′ arm of the stem loop. U1 and U2 snRNAs are used for 5′ splice site and branch point recognition in the canonical splicing pathway (>99% of all splicing events) while U11 and U12 perform the same roles in the minor splicing pathway (<1% of all splicing events). The recognition sites (5′ and 3′ splice sites and branch point) are different for each pathway, whilst this document generally refers to the major pathway, the same concepts can also theoretically be applicable to the minor splicing pathway.

5′ Splice Site Sequences:

5′ splice site sequences are either known in the art or can be readily determined. A 5′ splice site of a mirtron according to the current invention preferably comprises the sequence GU as the terminal nucleotides in the 5′ to 3′ direction.

Preferably, the first four nucleotides of the 5′ splice site should not form a perfect complement with the corresponding 3′ nucleotides within the stem loop and should not deviate too much from a human consensus splice site sequence (GUN(A/G)G). Ideally, there should be two nucleotide mismatches or bulges to increase the chance of splicing:

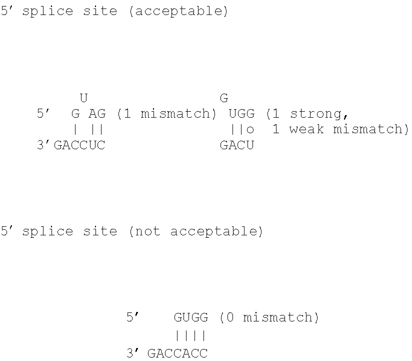

Therefore, a mirtron according to the current invention preferably comprises a sequence in which the first four nucleotides of the 5′ splice site do not form a perfect complement with the corresponding 3′ nucleotides in the stem loop or hairpin. Corresponding nucleotides are nucleotides that meet each other and have the potential to form base-pair interactions in the stem loop or hair-pin structure. Preferably there are 2 or more mismatches or bulges.

When the invention is for use in humans, the nucleotides of the 5′ splice site should not deviate too much from a human consensus splice site sequence (GUN(A/G)G). Preferably therefore, the first four nucleotides of the 5′ splice site in the sequence of the mirtron are GUN(A/G)G.

Branch Point:

The human consensus branch point sequence is YUNAY, where Y is C or U and N is any nucleotide (Gao K, Masuda A, Matsuura T, Ohno K. Human branch point consensus sequence is yUnAy Nucleic Acids Res. 2008 Apr;36(7):2257-67), and it is typically found at least 10 nucleotides away from the 3′ splice site. The 5 nucleotides surrounding the branch points should not have more than 3 nucleotides that base-pair with the opposite arm of the stem loop to allow U2 to compete for binding and recognition. We have designed the branch points to be within the hairpin loop because of ease of design, but the branch point can also be located in the 3′ arm of the mirtron as long as it follows the above rules. An illustration of these requirements is shown below:

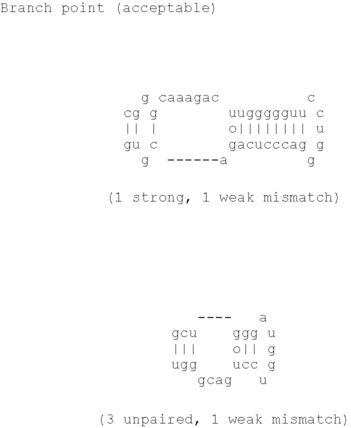

A mirtron according to the current invention therefore comprises a branch point sequence. The branch point sequence may be located within the hairpin loop or within the 3′ arm of the stem loop. The branch point sequence may be located at least 10 nucleotides away from the 3′ splice site, for example at least 15, 20, 25, 30 or 40 nucleotides away from the 3′ splice site. Preferably, out of the 5 nucleotides that surround the branch point only 1, 2 or 3 nucleotides base-pair with the opposite arm of the stem loop. More preferably, the branch point sequence corresponds to the human consensus branch point sequence and is YUNAY, where Y is C or U and N is any nucleotide.

3′ Splice Site Sequence:

3′ splice site sequences are either known in the art or can be readily determined. The almost absolute requirement for a 3′ splice site to be recognised is that it ends with an AG (Zhang M Q. Statistical features of human exons and their flanking regions. Hum Mol Genet. 1998 May;7(5):919-32). Therefore, a mirtron according to the invention preferably comprises a 3′ splice site sequence in which the terminal nucleotides are AG in the 5′ to 3′ direction.

Polypyrimidine Tract

Using rational design of mirtrons in Example 1, it was established that the 3′ polypyrimidine tract leading into the 3′ splice site must be greater than 15 nucleotides long. The mirtron sequence according to the invention therefore comprises a 3′ polypyrimidine tract of 15 or more nucleotides in length, for example 18 or more, 23 or more, or 28 or more nucleotides in length.

“Polypyrimidine tract” means that a series of nucleotides are present in the RNA sequence substantially consisting of pyrimidines, i.e. cytosine (C) and uracil (U). The tract may not consist entirely of pyrimidines. Preferably, it consists of greater than 70%, 80%, 90% or greater than 95% pyrimidines.

The polypyrimidine tract is located between the 3′ splice site and the branch-point on the 3′ arm of the stem loop.

Recognition by DICER

The second set of requirements for a mirtron comes from the need for DICER to recognize the processed intron as a substrate. In the endogenous RNAi pathway DICER recognises pre-miRNA structures characterised by a dsRNA stem of around 21-23nts and a hairpin loop at one end. As such, following the actions of lariat debranching enzyme, the spliced mirtron must show sufficient base-complementarity between the 5′ and 3′ regions for a stem to form. Synthetic shRNAs that mimic pre-miRNAs have been designed with 100% base-pairing in this stem, and also with every other nucleotide paired (see below).

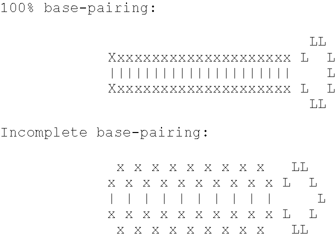

The mirtron sequence according to the current invention is therefore sufficiently base-complementary in the 5′ and 3′ region in order for a stem loop structure to form. The % complementarity is preferably greater than 50%, for example, greater than 60, 70, 80, 90 or greater than 95%.

It therefore seems that DICER is fairly flexible in its substrate specificity and the hairpin formed from the mirtron will have little limits over than being of the correct length. As long as the hairpin is processed by DICER then the dsRNA will be shuttled into RISC for silencing purposes. The constraints and outcomes of this are discussed in the following sections.

To date we have tested three different backbones for mirtron design based on the predicted mirtrons, mmu-miR-1224, hsa-miR-877 and hsa-miR-1226. Of these miR-1224 is 85nts in length, miR-877 is 86nts in length and miR-1226 is 75nts in length. All three are spliced out and we have confirmed knockdown of an artificial luciferase target for each (FIG. 3 and data not shown).

The crucial determinants for recognition by DICER will be the length of the dsRNA duplex that is required for RNAi and the length of the hairpin that is to be used. Research over the past 10 years has found that dsRNA delivered as an siRNA i.e. with no hairpin, can be a potent trigger for RNAi. Crucially it has been found that siRNAs designed with a 19nt region of base pairing and 2nt overhangs at each 3′ end (see below) are the most effective length of siRNAs in mammalian cells (Elbashir et al. 2001), although longer siRNAs and blunt ended siRNAs have been designed and shown to be effective.

| | | | |
|---|---|---|---|
| 3′ | TTXXXXXXXXXXXXXXXXXXX | 5′ | Sense |
| 5′ | XXXXXXXXXXXXXXXXXXXTT | 3′ | Anti-sense |
| | 19nt | | |

Endogenous miRNAs also share a preference for 2nt 3′ tails following processing by DROSHA and DICER whilst variation in the length is seen with the detection of anti-sense strands showing sizes ranging from 21-23nts (inclusive of overhang).

Analysis of predicted mammalian mirtrons suggests that unlike miRNAs and siRNAs there appears to be a preference of single-nucleotide overhangs at the 5′ and 3′ ends of the stem-loop structure, although a 2nt overhang is seen at the 3′ end of the 5′ arm species following processing by DICER. However the length of the anti-sense species produced falls into the same 21-23nt group as seen with miRNAs.

Taken together it is reasonable to suggest that the minimum number of nucleotides that are involved in the dsRNA duplex of the mirtron is likely to be approximately 30nts. The maximum number could be as large as 120 nts since synthetic shRNAs have been designed with multiple siRNA sequences placed one after another followed by a hairpin such that 3 different active RNAi species are produced from one hairpin (Sano et al. 2008). Introns are commonly found greater than 200nts in length, and as such, an approach mimicking these findings could be used as a mirtron as long as splicing constraints within the sequence are maintained. The length of the dsRNA stem of the mirtron may therefore be from 30 to 120 nucleotides long, for example from 45 to 75, 50 to 70, or from 55 to 65 nucleotides long.

With regards to the hairpin region, numerous hairpin sequences exist within the known miRNAs. There is much variation in length ranging from 48 (hsa-miR-320) to 150nts (hsa-miR-1302). Each of these hairpins acts as a substrate for DICER processing and thus could be mimicked in a synthetic design of a mirtron. In addition to this, synthetic designs have incorporated short hairpins with as few as 3nts present in the loop region and have been demonstrated to work. Therefore, the hairpin or loop region may be of any length, for example 3 to 150, 10 to 125, 20 to 100, 30 to 75 or 40 to 50 nucleotides in length.

Taken together it can be predicted that mirtrons may be functional when in the size range of approximately 45 to approximately 200 nucleotides in length. A mirtron according to the invention may therefore be from 45 to 200 nucleotides in length, for example 50 to 175, 75 to 150 or 100 to 125 nucleotides in length.

Antisense Sequence

In the mirtron sequence of the invention, the antisense sequence is selected or designed based on the sequence of the target gene. The following rules have been used for anti-sense designs.

Essentially in the first instance siRNAs may be selected based on the criteria that if they have a 5′ mirtron consensus sequence they can be placed on the 5′ arm, or if they are pyrimidine-rich they can be placed on the 3′ arm. If such siRNAs are found and are predicted to be good siRNAs, based on the criteria set out below, then they are incorporated into the backbone of the mirtron with the anti-sense strand remaining largely un-altered but the sense strand being altered to incorporate mirtron requirements (for splicing and DICER recognition as discussed above).

Rules for Target Selection:

The selection of siRNA against a gene of interest starts with an annotated target mRNA sequence including its 5′ and 3′ un-translated regions and its splice, polymorphic and allelic variants. Because the coding sequence is the most reliable mRNA sequence information available, it is commonly targeted. The UTRs are generally less well characterised but can also be targeted with similar gene knockdown efficiency. It is recommended to avoid targeting sequences that contain known binding sites for mRNA-binding proteins such the exon-exon junction complex. Additional considerations can be made in identifying siRNAs that target orthologs in more than one species or all splice variants of a gene.

Initially a search can be made in databases which are available that archive experimentally tested siRNA sequences from the literatute (http://sirecords.umn.edu/siRecords/ or http://sirna.cgb.ki.se/). Validated siRNAs can also be acquired from commercial resources. If siRNAs are to be designed then it is advisable to select 3-5 candidate siRNAs using available guidelines and tools.

Several siRNA sequence selection algorithms have been developed (http://sirna.cgb.ki.se/ and http://jura.wi.mit.edu/bioc/siRNA). A small number of these algorithms will also consider the secondary structure and accessibility of the targeted mRNA which may affect efficiency (http://www.cs.hku.hk/˜sirna/ and http://sfold.wadsworth.org/index.pl). However, if sequences are to be designed without the aid of a design programme or registered supplier, the following rules are advisable where possible to ensure increased specificity and efficacy, and these are incorporated into design algorithms^{.}

21nt in length with symmetric 2nt 3′ overhangs (i.e. strand length is 19nt and at the 3′ end of each strand there is a 2nt overhang, typically TT). Although longer dsRNAs appear to have the advantage that they can be transfected at lower concentrations than conventional siRNAs without loss of gene silencing, they also appear to be more likely to induce non-specific responses or mediate other effects on cell viability.

Primary sequence asymmetric (different nucleotides at each end).

A and U enriched 5′ ends of the anti-sense/guide strand e.g. U or A at position 1 of anti-sense/guide strand, A and U richness in positions 1-7 of the anti-sense/guide strand.

G and C enriched 3′ ends of the anti-sense/guide strand e.g. C or G (more commonly C) at position 19 of the anti-sense/guide strand.

G and C content of 30-55% (from analysis of functional siRNAs). Too low may destabilise the siRNA duplex and reduce affinity for target mRNA binding. Too high and RISC loading/cleavage may be impaired.

Avoid internal repeats or palindromes which could form secondary intra-strand secondary structures that can interfere with the RNAi process.

Design low internal stability around positions 9-14 e.g. A or U at position 10 of the anti-sense/guide strand. The A or U at position 10 is at the cleavage site and is believed to promote catalytic RISC-mediated passenger strand and substrate cleavage.

Extended runs of altering G and C pairs (more than 7) or runs of more than three guanines should be avoided.

Filter out siRNA sequences containing putative immuno-stimulatory motifs in either strand to minimize toxicities and non-specific silencing effects.

The current hypothesis is that the strand with the less stable 5′ end, owing either to weaker base pairing or introduction of mismatches, is favourably loaded into RISC. The above rules agree with this hypothesis of thermodynamic asymmetry and may contribute to the bias for selection of the anti-sense/guide strand in RISC. Chemical methods of preventing passenger-strand use have also been introduced and can be used (e.g. Dharmacon's ON-Target™ siRNA). It is important to design a siRNA with these rules such that the anti-sense/guide strand is incorporated preferentially.

Following the design process each candidate siRNA should be examined for similarity to all other mRNA transcripts that might unintentionally be targeted at a genome-wide level. Each strand of an siRNA duplex, once assembled into RISC, can guide recognition of fully and partially complementary target mRNAs, referred to as ON and OFF targets respectively. Identifying possible OFF-targets can be achieved by entering the complementary siRNA strand sequences into a BLAST search and looking for sequence homology. Particular attention should be paid to positions 2-8, the seed region, which has a major role in siRNA specificity. It is advised that at least 3 mismatches should be made between positions 2 and 19 from OFF-targets and the mismatches near the 5′ and in the centre of the examined strand should be assigned higher significance. In contrast anti-sense/guide strand position 1 and nucleotides at 3′ overhangs have little, if any, contribution to the specificity of target recognition.

Within the endogenous RNAi pathway two actions can be performed depending on the degree of base-pairing between the mRNA target and the anti-sense strand of the active RNAi species. Complete base-pairing directs mRNA cleavage whereas the predominantly encountered partial base-pairing, including nucleotides 2-9 that form the seed region that is dis-proportionally involved in target binding (Haley et al. 2004), directs translational repression.

The first case of complete base-pairing is what is commonly aimed for when RNAi is exploited synthetically as in most cases elimination of the mRNA transcript is desired. However the endogenous miRNAs follow the second scenario of incomplete base-pairing, and as expected, a synthetic construct, be it a siRNA, shRNA or miRNA mimic, can be designed to have this effect. The result is that the transcription of the mRNA is repressed, so although it is still present the protein is not produced and you effectively see gene silencing. Furthermore, in the endogenous pathway miRNAs actually target the 3′ untranslated region of the mRNA transcripts whereas synthetically designed constructs normally target the open-reading frame that is coding. The reason miRNAs target the 3′UTR and not the ORF appears to be so that the necessary proteins required for repression are not blocked by the ribosomal proteins translating the mRNA.

Therefore, according to the current invention the mirtron sequence comprises an antisense sequence that is at least partially complementary to a sequence in the target mRNA sequence. It may be designed to have complete complementarity to the target mRNA sequence to effect elimination of the mRNA transcript. Alternatively, the mirtron may be designed with partial complementarity to the target mRNA in order to direct translational repression. A partially complementary antisense sequence may be partially complementary in relation to the entire length of the designed antisense sequence. Alternatively, partially complementary may be limited to the 2-9 nucleotide seed region. Partial complementarity may be at least 40, 50, 60, 70, 80, 90 or at least 95% complementary. The antisense strand may be located on the 5′ or 3′ arm of the mirtron.

The mirtron may target the 3′UTR or the ORF of the mRNA. When targeting the 3′ UTR it is necessary to make sure that the seed region that lies at nucleotides 2-9 of the anti-sense strand pairs to the target. When designing constructs we generally look for 8 nucleotide sequences repeated in the 3′UTR to maximise our chances of success. A suitable programme can be used to identify these repeats together with all acceptable variants that can be accommodated.

In addition, the backbone to the synthetic mirtron must be considered carefully. Using one such as miR-877 which uses its 5′ arm as the anti-sense strand would be difficult to incorporate this strategy since the 5′ consensus splice sequence would overlap with the seed region and immediately specificity would be reduced since only 4nts of the seed region would be different for each target. Instead, a mirtron such as miR-1226 could be used to incorporate this design since the 3′ arm is dominant according to small RNA libraries. In this way the seed region is now placed immediately after the hairpin and some 12-20 nucleotides, at least, from the 3′ splice site. Without a constraint of the seed sequence other than that pyrimidine rich would be ideal, there is much more scope for targeting genes specifically and reducing off-target effects.

Mammalian Mirtron Sequences

Known or predicted mammalian mirtron sequences can be used as a template for designing the mirtron to target the gene or other sequence of interest. Examples of mammalian mirtrons are provided below.

The following sequences are predicted mammalian mirtron sequences (Berezikov et al. 2007):

miR-877
(SEQ ID NO: 1)
GTAGAGGAGATGGCGCAGGGGACACGGGCAAAGACTTGGGGGTTCCTGG
GACCCTCAGACGTGTGTCCTCTTCTCCCTCCTCCCAG
miR-1224
(SEQ ID NO: 2)
GTGAGGACTCGGGAGGTGGAGGGTGGTGCCGCCGGGGCCGGGCGCTGTT
TCAGCTCGCTTCTCCCCCCACCTCCTCTCTCCTCAG
miR-1225
(SEQ ID NO: 3)
GTGGGTACGGCCCAGTGGGGGGGAGAGGGACACGCCCTGGGCTCTGCCC
AGGGTGCAGCCGGACTGACTGAGCCCCTGTGCCGCCCCCAG
miR-1226
(SEQ ID NO: 4)
GTGAGGGCATGCAGGCCTGGATGGGGCAGCTGGGATGGTCCAAAAGGGT
GGCCTCACCAGCCCTGTGTTCCCTAG
miR-1227
(SEQ ID NO: 5)
GTGGGGCCAGGCGGTGGTGGGCACTGCTGGGGTGGGCACAGCAGCCATG
CAGAGCGGGCATTTGACCCCGTGCCACCCTTTTCCCCAG
miR-1228
(SEQ ID NO: 6)
GTGGGCGGGGGCAGGTGTGTGGTGGGTGGTGGCCTGCGGTGAGCAGGGC
CCTCACACCTGCCTCGCCCCCCAG
miR-1229
(SEQ ID NO: 7)
GTGGGTAGGGTTTGGGGGAGAGCGTGGGCTGGGGTTCAGGGACACCCTC
TCACCACTGCCCTCCCACAG
miR-1230
(SEQ ID NO: 8)
GTGGGTGGGGGCATCTCGGAGGAGGTGGGGGGTGTGGCGCCCAGCGGAT
GACTCCGAGCGGCTCCTTTCCCAG
miR-1231
(SEQ ID NO: 9)
GTCAGTGTCTGGGCGGACAGCTGCAGGAAAGGGAAGACCAAGGCTTGCT
GTCTGTCCAGTCTGCCACCCTACCCTGTCTGTTCTTGCCACAG
miR-1232
(SEQ ID NO: 10)
GTGGGGTGGCGGCGACATGGCGGGGGCGGCGGGCCCTGCGGAGGCTGTG
CGCCTGACCCCGACCACCCCGCAG
miR-1233
(SEQ ID NO: 11)
GTGAGTGGGAGGCCAGGGCACGGCAGGGGGAGCTGCAGGGCTATGGGAG
GGGCCCCAGCGTCTGAGCCCTGTCCTCCCGCAG
miR-1234
(SEQ ID NO: 12)
GTGAGTGTGGGGTGGCTGGGGGGGGGGGGGGGGGGCCGGGGACGGCTTG
GGCCTGCCTAGTCGGCCTGACCACCCACCCCACAG
miR-1235
(SEQ ID NO: 13)
GTGGGCCTGGGTCGGTGGGGACGGGGCGGCTGGGCGTGCCCTGCGGCCG
CTGCTCTAACCGCACCGTCCCCCAG
miR-1236
(SEQ ID NO: 14)
GTGAGTGACAGGGGAAATGGGGATGGACTGGAAGTGGGCAGCATGGAGC
TGACCTTCATCATGGCTTGGCCAACATAATGCCTCTTCCCCTTGTCTCT
CCAG
miR-1237
(SEQ ID NO: 15)
GTGGGAGGGCCCAGGCGCGGGCAGGGGTGGGGGTGGCAGAGCGCTGTCC
CGGGGGCGGGGCCGAAGCGCGGCGACCGTAACTCCTTCTGCTCCGTCCC
CCAG
miR-1238
(SEQ ID NO: 16)
GTGAGTGGGAGCCCCAGTGTGTGGTTGGGGCCATGGCGGGTGGGCAGCC
CAGCCTCTGAGCCTTCCTCGTCTGTCTGCCCCAG
miR-1239
(SEQ ID NO: 17)
GTGGGTGGGCAGGTGGGTGGGAAGCCCTGGGACGCTGCCTCCTCTCTCC
TGGGGCCTCTCTCGGGCTGGGGGCTGGTCTCAGTTTCCCCATTCTGCCT
GGCCTAG
miR-1240
(SEQ ID NO: 18)
GTGGGCCAGGGCCGCGGGGGGGAGCAAGCCATCTAGCATTCCTGGGAAA
CGCTTACATCTCACCATGACCCTGATCCCACTAG
miR-1241
(SEQ ID NO: 19)
GTGAGGGGGCTGGCATGGCGAGGAGGCGCCAGAGAAGCCATAGTGTGGG
GATGGGCTGCACACTCACCTCTCTGTGCCTTCCAG
Other potential mammalian mirtrons include:
unnamed macaque mirtron
(SEQ ID NO: 20)
GTGAGTCTGGGTGGGGTGCAGGGCCGGCGGGTGTGGGCTGTGGGCAGCA
GGTAGAGGCAGACAGTCACCCTGAACCCCGTCTCTCCCATCTGCCCACC
GTCAG
unnamed macaque mirtron
(SEQ ID NO: 21)
GTGAGCTTGGCGGGGCTGCTGGAGGAGTGGGTTCGCCCAGTCTGGGCAC
CAGACACGGAGACTCCAGCCCACCTCCTTCTCCCCAG
unnamed human mirtron
(SEQ ID NO: 22)
GTGGGTGGGCTGGGCGGGGGGCGGGGCAGGTGGGCGGAGGCCCTGGGGC
TCTGCATAGCAGCAGCCCCATGCCCCACCTCCCTGTCCCAG
unnamed human mirtron
(SEQ ID NO: 23)
GTGAGTGGCCACCATGCGGGGACAGGGGCAGGGGCAGCCCTCACCCACA
GCCTCTCACCTGCCTTTGTCCACCCACAG
unnamed chimp mirtron
(SEQ ID NO: 24)
GTGAGGGCAAGGCTGGGGGGCCCCTGGGCTAAGTGGGAGCCTGGCTGGA
ATTCCCACTCCACCTTACTCTCCTGCAG
unnamed human mirtron
(SEQ ID NO: 25)
GTGAGTGTGGGGCGCGCCGGGCTGTGGCGGGCTGGGGGCGGGCGGCCCT
GGGTCCCAGCCTCCTGCTGCCCACCGCTGCCCACCGCAG
unnamed macaque mirtron
(SEQ ID NO: 26)
GTGAGCAGAGTGGGTTGGAGGGGGGTGTCCCAGGCTCTTTCTTGTCTAT
GGGCCTGACACCCCGACCCTGACTGGCCTGGGCCTCCCAG
unnamed macaque mirtron
(SEQ ID NO: 27)
GTGAGGAGGCAGGCGGGGAATGCCTGAGCCGCAGGGGGCCTGGGCCTGG
ATCCCAGCCGGCCCAGATTTATTTTCATCTCCTGCTTCCTGCCAG
unnamed mouse mirtron
(SEQ ID NO: 28)
GTAGTCTGGGAGCTGGCACCGCAGACTATCCCCAGGGGACACGGGGACT
TGGCTGAGGCAGCTCCACTGAGAGCTGAGAGCCTTATCCCCGCAG
unnamed mouse mirtron
(SEQ ID NO: 29)
GTAAAGGCTGGGCTTAGACGTGGCCTTTGGGTGTGGAATGCACTTCCGT
TTGTAACCGCCATCTAACCCTGGCCTTTGACAG
unnamed human mirtron
(SEQ ID NO: 30)
GTGAGCTGGGGTGGCTGAGGCGGGATGGGGGCCACCTGAGGCTGGGGCT
GGCCCTGCTCACTGCTGCTCCTGCCCACAG
unnamed human mirtron
(SEQ ID NO: 31)
GTGGGCGAACGCCCGGGTGGAGCGGGTCAGGCAGGGCCAGTCACAGACA
CAGCTCTGTGCTGACTGCCCGCCTTGCCCACAG
unnamed human mirtron
(SEQ ID NO: 32)
GTGAGCAGGGCCAGTGCTGGACTCTGCTGCTGGGCCTTGGTGGGCAGGG
GCAGCAAGAGAGGTTCAGCTTTGGCCCTGGCCCAGCTCTCTGGGCACAG
GGGTCAGCTGGGATGGGAGTCTGGAGAACAAGGGGTCCCTGAAGACCAG
CAACCATCTCACTCTGGCACCCCTGCTCCCCCAG
unnamed human mirtron
(SEQ ID NO: 33)
GTAGGGCCCCGCCGAGGGGGCAGGGTGGGGGCCCCAGGGACCCCCCTCA
CGGCCTGCGGTCTGGGCTCTCGGCAG
unnamed human mirtron
(SEQ ID NO: 34)
CCAGAGTGCGTGAGGACACGTAGAGGGGCTCAGGCTGCCAAGGGGGCAC
GGAGCTTGTGGGAGCACCAGGACTGGGACTGTTCATGTGGGTGTCCTCT
GCCCTCCCTGACCCCTTGCCTGTCTCTCGCCACAG
unnamed human mirtron
(SEQ ID NO: 35)
GTAGGCAGAGGGGCAGGGTGGTGGCGGGGGAGAGGGTAGGGGGGCGGG
GCCGCAGTGCTCAGCTGTCTTCCCCTCGGCCCTGCCCCACAG
unnamed human mirtron
(SEQ ID NO: 36)
GTGCGTGGTGGCTCGAGGCGGGGGTGGGGGCCTCGCCCTGCTTGGGCCC
TCCCTGACCTCTCCGCTCCGCACAG
unnamed human mirtron
(SEQ ID NO: 37)
GTGAGGGCAGCCGGCAGGGCCCCAGGTCCTGCTTACATGTGGGCCCAGA
CTCCAGCTCCCTCTCCCCACATGCAG
unnamed human mirtron
(SEQ ID NO: 38)
GTGAGGCGGGGCCAGGAGGGTGTGTGGCGTGGGTGCTGCGGGGCCGTCA
GGGTGCCTGCGGGACGCTCACCTGGCTGGCCCGCCCAG
unnamed human mirtron
(SEQ ID NO: 39)
GTGAGTGGGGTGGGGGTGTGGGGTGGGGGGCATGGAGCCGGCGTGGAAC
CAGAGCCCTCACTCCTGCCCACACCCCTCAG
unnamed human mirtron
(SEQ ID NO: 40)
GTGAGCGGCCGCTGGGGGCAGGTGGCGGGTGGGAGGGAGGGAGGTGGGC
TTTCCCGGTGGGCGTTCAGAAACCCCCTTTTACCTGACTCTCTCCCAG
unnamed human mirtron
(SEQ ID NO: 41)
GTGAGCCAGTGGAATGGAGAGGCTGTGGGCAGGGGGAGATGTGAAGGAA
AGAACTAGGACCCATTCATCCACTGCATTCCTGCTTGGCCCAG
unnamed human mirtron
(SEQ ID NO: 42)
GTGAGGCTGGGGCAGGGATGAGGTGGCCCAGGAGAGTGAGTCCCCGAGG
GTGGGCCGCGGAAAGCCAGCAGGCTGACCAGGCCTTCGTGTCCCCTCCC
TGCCAG
unnamed human mirtron
(SEQ ID NO: 43)
GTAGGGGCGGGGTGGGAGAGGTGGGGTCTGCGTGCGGGCCGGGCCGGCC
CCTGACGCGCGCTCCTCCCCTGCCCCAG
unnamed human mirtron
(SEQ ID NO: 44)
GTGGGTGCGGAGGCGGGGGGACAGGGTACCCACGAGGCCTGAACTCACA
CCTCGGCGCCTCCTGGCCCCGCAG
unnamed human mirtron
(SEQ ID NO: 45)
GTGAGCCCGCAGCGGGGCAGGGTACAGGGGCGGGGGCCCCCATGGCCAG
GCCCCACCCCGCTCCTATTGACCCTCCTGCTGTCCTCAG
unnamed human mirtron
(SEQ ID NO: 46)
GTGAGGGGGCTGCCAGGGGTAGGCTACAGGCCTCCATCACGGGGGACCC
CTCTGAAGCCACCCCCTCCCCAG
unnamed human mirtron
(SEQ ID NO: 47)
GTGAGGGGCTGGGGTTTTCGTCCCATGGCGGGCAGGGCCCAGGGAGCTG
AGGCTGCTTCTAAAGCCATCTCCCCGGTACCCGCAG
unnamed human mirtron
(SEQ ID NO: 48)
GTAGGTCAGGCCCAGGTGTGGGTTGGGGTCCGGCTGCAGCGCCCGCTAA
CCCACCTGCTGCTGTCCAG
unnamed chimp mirtron
(SEQ ID NO: 49)
GTGAGTGGGCAGGGACAGGAGCCTGCGGGCTGGGGTTGGGGGGCACAGT
GGGTGTGCCCGCTTGGCCTGTGACCACCTGCACTGTGCCCCAG
unnamed chimp mirtron
(SEQ ID NO: 50)
GTGAGTGGGGGCTTCAGGCTTCAGGGCGGGGGGGAGGGGAACGCCGGAG
GCCCCTCCGCGGCCCTCACCTACCCTCCCCCGCCAG
unnamed macaque mirtron
(SEQ ID NO: 51)
GTGAGCAGGGATGGCGGGCATTGGGGGAGCCGGGAGCGGGGGACAGCCG
GGGTCCCTCTCACGCCCGCCTCGCTGCCCGGCAG
unnamed macaque mirtron
(SEQ ID NO: 52)
GTGAGCACGCGGGATTTCCAGGGGACGGGCAGGACGCTGCAGTCTGACT
GGCGCAGGGGCCTGGGAATCCGCCTGTCTCCCACAG
unnamed macaque mirtron
(SEQ ID NO: 53)
GTACGGAAGGCCAGGGTGGTGGCGGGAATCCATTCCGGTGTGTTCTACA
CATTGCTTAGAGATTGGATGGGGTGCATCAGGGTGGTTTCTGATTACTT
TTCTTCTTCCCTCAG
unnamed macaque mirtron
(SEQ ID NO: 54)
GTGAGCTGGGGGATGTGAGGGGTTGGACTGTTCCAGTTCTCTGTCTACC
CTCTCTGCCTCCTGCCTGCAG
unnamed macaque mirtron
(SEQ ID NO: 55)
GTGAGGCGGGCAGACGGCATGGGAGGGCAGGCGGGCTGGGTTTCTGACT
GGCCCTGGCTGACCGTGCTTCCCTCTTCCCTCTGCCCCGCCCAG
unnamed macaque mirtron
(SEQ ID NO: 56)
GTGAGGCCGACGCGAGGCGGCTGCGAGACCCCGTGCGGCCTTGCGGGCG
CGCCCTCACCCTCTGCCGTCCCTGTCTCACAG
unnamed macaque mirtron
(SEQ ID NO: 57)
GTGAGTGTGGGGTGGCTGGGGTGGGGCAGGGATGGCTTGGGCCTGCCCA
GTCGGCCTGACCACCCACCCCACAG
unnamed macaque mirtron
(SEQ ID NO: 58)
GTGAGCAGGGCGATCGGGGGGGCTGGCGTGCTGGGGTGGGGCACAGGGG
GTGCCTCAGCCGCCTCTGAGCTCCCACAACCCGCCCCTGCCAG
unnamed macaque mirtron
(SEQ ID NO: 59)
GTGAGTAGGGGCGGGAGGAGGGGACACCCTGAGCTCAGTCCAGCCAGGA
GAGGCATTGGGGTGTCCCTGCCATCTGGCCGTGGAGGGGCAAGGCAGGA
GGTATGTGCCATGGGTGGGGGCAGTTGAGCTGAGGTAGGAGGTGACCCT
GCCTGCCACCTGCCTTTAG
unnamed macaque mirtron
(SEQ ID NO: 60)
GTAGGTGCGGCGGTGCGGCTGTGGGAGGGCTGCCGGTGGCGCTCCCTCT
GAGCCTGCCGCCTCGCAG
unnamed macaque mirtron
(SEQ ID NO: 61)
GTGGGAGCAGGCAGGGCTGGGCAGGCCAGGGGTCTGGGAGGCAGGGGCC
CGGGCAGCCCTGAACCCAACCCCTGGCTTCCTCTCCCATCCCAG
unnamed macaque mirtron
(SEQ ID NO: 62)
GTGAGCCAGAGTGGTGGAGCAGGGAGGCGGGGGTTCAGCCAGGGCTTGC
TGGGCCAGCCAGAGCGCCCCCGTGATGCCCTGCGGCTCTGTTCTCACAG
unnamed mouse mirtron
(SEQ ID NO: 63)
GTGAGAGCGGGGGGAGTGGCAAGTGAGACGGATGGGCGGGGCTGGCTCA
GACTGCTAGCCCCGCCCAGCTCTCTAACTCTTCCCTTGTGCCCTCAG
unnamed mouse mirtron
(SEQ ID NO: 64)
GTGAGGCTCAGTATGGGGTGGGGGTGTCGTCGCCTGCCCGACTGACCAC
CCACTCACCCTGGACTGACTCTCAG

Therapeutic Treatment and Strategies

The mirtrons of the current invention are for use in modifying expression of a gene in a mammalian cell. They may be used as research tools or have a therapeutic purpose. The mirtrons of the invention may be used to treat any disease which can be treated by gene silencing or knock down. As used herein, the term “treat” or “treatment” is meant to encompass therapeutic, palliative and prophylactic uses.

The invention provides a mirtron or gene comprising a mirtron as defined herein for use in the treatment of a disease which can be treated by gene silencing or knock down, i.e. for use in the treatment of a genetic disease by reducing or eliminating the expression of a target defective gene. Preferably, the target gene comprises one or more dominant gain-of-function mutation(s). The invention also provides a method of treating an individual for a disease which can be treated by gene silencing or knock down comprising administering a mirtron or a gene comprising a mirtron as defined herein to said individual.

Gene Knockdown and Replacement Strategy:

Many diseases are the result of toxic gain of function mutations or gene overexpression. At present there are relatively few approaches to tackle the underlying cause of the disease. In the case of autosomal dominant mutations, allele-specific silencing can sometimes be applied in which the sequence specificity of RNAi is exploited such that the mutant allele is knocked down whilst the wild-type is retained. However, often the identification of appropriate species capable of providing enough discrimination for the approach to be successful is both exhaustive and expensive. In addition, multiple mutations in the same gene may have the same disease phenotype meaning multiple RNAi species must be identified to treat the same disease in different patients. In the case of gene over-expression as a result of promoter abnormalities or gene locus triplication then the only approaches to tackle the phenotype is to reduce levels of protein activity through drug antagonists that do not tackle the underlying cause, or regulating gene expression using RNAi. In some cases it has been reported that the complete ablation of the gene may be tolerated and lead to phenotypic benefit but it is highly unlikely that such an approach is valid for all such diseases given the importance of the gene in adult life.

An alternative approach has been investigated using siRNAs and shRNAs in which gene knockdown is coupled to gene replacement (Kim et al. 2003). In such a strategy an anti-sense sequence is used to target the gene of interest at a site not incorporating mutations such that both wild-type and mutant alleles are ablated. Concomitantly a modified version of the gene of interest is delivered with specific mutations made in the anti-sense target site. These do not affect the protein sequence but make the replacement mRNA resistant to silencing. To date the approach has been explored as a potential treatment for ALS where the mutant form of copper, zinc superoxide dismutase is toxic but the wild-type allele is indispensable (Xia et al. 2005), and for retinitis pigmentosa where 25% of all cases are caused by over 100 different mutations in the rhodopsin gene (Kiang et al. 2005, O'Reilly et al. 2007).

In one aspect of the invention, the mirtron, or gene or construct comprising the mirtron, targets the gene of interest, thereby resulting in gene silencing or knockdown. A replacement gene may be administered at the same time as the mirtron, thereby achieving simultaneous knock-down and replacement. For example, a single construct may comprise both the mirtron and the replacement gene. The mirtron is preferably positioned within the replacement gene, e.g in place of a natural intron. Alternatively, the mirtron and replacement gene may be administered separately or sequentially to achieve the same aim.

In one embodiment, the replacement gene is a modified version of the wild type gene of interest. The replacement gene may comprise one or more mutations in the antisense target region of the mirtron. This prevents the replacement gene from being down-regulated, in addition to the target defective gene, by the mirtron. In this strategy, the sequence of the replacement gene is altered such that it is not recognised by the mirtron but is still a functional gene.

The approach has the advantages over the currently existing siRNA/shRNA strategy that:

1. The generation of the active species may bypass both Drosha and Exportin-5

2. Delivery can be in the form of a DNA-encoded plasmid enabling long-lasting viral delivery

3. The size of the virus can be minimised since only one gene promoter is needed to drive expression of both trans-gene and mirtron

4. The expression can be performed under the control of the endogenous gene PolII promoter rather than a ubiquitous PolIII promoter predominantly used for shRNAs and miRNA mimics. Such an approach would lead to site-specific delivery of the trans-gene thus minimising off-target effects of the RNAi species and would additionally allow endogenous control of transgene and mirtron expression levels in accordance to their natural stimulants and inhibitors.

Examples of diseases and genes that could benefit from this strategy are now provided.

TABLE 1
Diseases and target genes

| DISEASE | GENE |
|---|---|
| DRPLA | ATN1 or DRPLA |
| (Dentatorubropallidoluysian atrophy) | |
| HD (Huntington's disease) | HTT (Huntingtin) |
| SBMA (Spinobulbar muscular | Androgen receptor |
| atrophy or Kennedy disease) | on the X |
| | chromosome. |
| SCA1 (Spinocerebellar ataxia Type 1) | ATXN1 |
| SCA2 (Spinocerebellar ataxia Type 2) | ATXN2 |
| SCA3 (Spinocerebellar | ATXN3 |
| ataxia Type 3 or Machado- | |
| Joseph disease) | |
| SCA6 (Spinocerebellar ataxia Type 6) | CACNA1A |
| SCA7 (Spinocerebellar ataxia Type 7) | ATXN7 |
| SCA17 (Spinocerebellar ataxia Type 17) | TBP |
| FRAXA (Fragile X syndrome) | FMR1, on the |
| | X-chromosome |
| FRAXE (Fragile XE mental retardation) | AFF2 or FMR2, on |
| | the X-chromosome |
| FRDA (Friedreich's ataxia) | FXN or X25, (frataxin) |
| DM (Myotonic dystrophy) | DMPK |
| SCA8 (Spinocerebellar ataxia Type 8) | OSCA or SCA8 |
| SCA12 (Spinocerebellar ataxia Type 12) | PPP2R2B or SCA12 |
| Marfan Syndrome | Fibrillin |
| Atopy | Interleukin-4 |
| | receptor alpha |
| myeloproliferative disorders | JAK2 |
| ALS | SOD1 |
| Parkinson's Disease | Alpha-synuclein |

### SPECIFIC EXAMPLES

1. Huntington Disease

The Huntingtin protein is a protein of unknown function that is essential in nerve cells. Huntington's disease is caused by expansion of CAG repeats in the Huntingtin gene, which results in a gain of toxic function and death of neurons. An estimated 3-7 per 100,000 people of European ancestry suffer from Huntington disease and the disease is adult-onset, which means therapeutic intervention with gene knockdown and replacement, given early diagnosis, can significantly improve prognosis. Lin et al. 1995 (Genomics. 1995 Feb 10;25(3):707-715.) have elucidated the promoter control regions for the Huntingtin gene in humans. Utilizing this knowledge, a construct containing the human huntingtin promoter region, a codon-replaced huntingtin protein gene and a mirtron inserted into a natural intron can be constructed into a lentivirus vector specific for neurons and delivered to the brain.

2. Myotonic Dystrophies

CCHC-type zinc finger, nucleic acid binding protein (CNBP) (type I) and dystrophia myotonica-protein kinase (DMPK) (type II) are essential genes mutated in myotonic dystrophy type I and II respectively. The disease is caused by the expansion of CCTG tetranucleotide and CTG trinucleotide repeats respectively, resulting in gain-of-function mutations. These diseases affect 1 in 8,000 people worldwide and are adult-onset, which means if we intervene early enough with gene therapy that both knocks down the mutant and replaces it with the wild type version of these genes, it may be curable. As for the Pol II promoter sequence' to be used, Strobeck et al. 1998 (J Biol Chem. 1998 Apr. 10;273(15):9139-47.) has elucidated the promoter control regions of the DMPK gene and Shimizu et al. 2003 (Gene. 2003 Mar. 27;307:51-62) have done likewise for the CNBP gene. Utilizing this knowledge, a construct containing the DMPK/CNBP promoter region, a codon-replaced DMPK/CNBP gene and a mirtron inserted into a natural intron position of DMPK/CNBP can be constructed into an adeno-associated virus vector specific for muscle and delivered to muscle.

3. Fibrillin in Marfan Syndrome

“The fibrillin-1 gene encodes the glycoprotein fibrillin, a major building block of microfibrils, which constitute the structural components of the suspensory ligament of the lens and which serve as substrates for elastin in the aorta and other connective tissues. Abnormalities involving microfibrils weaken the aortic wall. Progressive aortic dilatation and eventual aortic dissection occur because of tension caused by left ventricular ejection impulses. Likewise, deficient fibrillin deposition leads to reduced structural integrity of the lens zonules, ligaments, lung airways, and spinal dura. Production of abnormal fibrillin-1 monomers from the mutated gene disrupts the multimerization of fibrillin-1 and prevents microfibril formation. This pathogenetic mechanism has been termed dominant-negative because the mutant fibrillin-1 disrupts microfibril formation though the other fibrillin gene encodes normal fibrillin. This proposed mechanism is evinced by the fact that cultured skin fibroblasts from patients with Marfan syndrome produce greatly diminished and abnormal microfibrils.” (http://www.emedicine.com/ped/TOPIC1372.htm)

Marfan syndrome affects about 1 in 10,000 individuals and perhaps as many as 1 in 3000-5000 (http://www.emedicine.com/ped/TOPIC1372.HTM). It is a juvenile onset disease that affects development as well, which means gene therapy using the gene replacement / knockdown strategy can prevent further deterioration and may restore function but cannot ‘cure’ the disease. However, given the lack of available corrective therapy, this may be the best approach possible.

Unlike the above examples, the exact boundaries of the promoter region of fibrillin-1 is not known, but based on sequencing data from patients, it is possible to narrow crucial regions to within 20 kb of the fibrillin-1 gene. If further work was done to elucidate the essential regulatory sequences within that region, a minimal promoter could be constructed and cloned, as described above in example 1 into a adeno-associated virus vector for expression of fibrillin-1 in connective tissues and muscles.

4. Interleukin-4 receptor alpha in Atopy (diseases due to strong allergic reactions)

“Atopy is characterized by the formation of IgE antibody in persons with a genetic predisposition, who respond with immediate hypersensitivity on exposure to specific allergens. Atopy is common, affecting up to 40 percent of populations of Western societies and it underlies the development of allergic diseases in susceptible persons.” (Hershey G K, Friedrich M F, Esswein L A, Thomas M L, Chatila T A. The association of atopy with a gain-of-function mutation in the alpha subunit of the interleukin-4 receptor. N Engl J Med. 1997 Dec. 11;337(24):1720-5.)

A few gain-of-function alleles of Interleukin-4 receptor alpha (IL4Ra), an essential gene in immune cells, genetically predisposes patients to atopic diseases. If a patient is identified to have a gain-of-function mutation by genetic screening, replacement of the mutant allele with the common wild type allele will presumably alleviate the disease, or if detected early enough, prevent development of allergic diseases before occurrence. The promoter region of the gene has been characterized by Hasomi et al. 2004 (Hosomi N, Fukai K, Oiso N, Kato A, Ishii M, Kunimoto H, Nakajima K. Polymorphisms in the promoter of the interleukin-4 receptor alpha chain gene are associated with atopic dermatitis in Japan. J Invest Dermatol. 2004 March;122(3):843-5). Control of this gene is of utmost importance because over-expression of this disease can also cause atopic dermatitis, as detailed in Hasomi et al. Hence, the use of the endogenous promoter region of IL4Ra linked to a codon-replaced gene with a mirtron to knock down disease-causing alleles delivered to T cells by lentivirus vectors will allow for therapeutic intervention for atopy in susceptible individuals.

Simultaneous delivery of more than one intron-based therapy within a single mRNA molecule; Simultaneous delivery of mirtron and mRNA

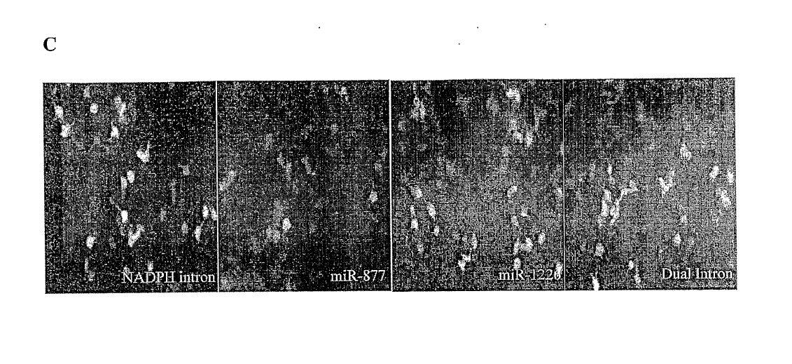

The current invention is not limited to delivery of a single intron-based therapy within a single mRNA molecule. On the contrary, multiple intron-based therapies may be delivered, either within the same mRNA molecule or within more than one mRNA molecule. Such combinatorial therapy allows the targeting of multiple genes/proteins simultaneously. This may be useful for example where multiple defective genes result in a disease or condition. An example of this is demonstrated in a dual intron vector which contains miR-877 and miR-1226 as separate introns in an eGFP mRNA (FIG. 10A). Knockdown of the miR-877 and the miR-1226 targets are at levels comparable with the single intron vectors and this is not dependent on off-target effects (FIG. 10B). As demonstrated by eGFP fluorescence, splicing of the mRNA is not affected by the addition of additional mirtrons, which suggest that multiple mirtrons can be produced from the same mRNA without compromising on transcription, processing and translation of the mRNA.

Furthermore, it is possible to simultaneously deliver the mirtron to effect silencing of the gene of interest and an mRNA coding for a useful protein. For cancer therapy for example, a mirtron for knockdown of an oncogene in a particular cancer may be codelivered with a gene that codes for a pro-apoptotic gene such as E1A (Opalka B, Dickopp A, Kirch H C. Apoptotic genes in cancer therapy. Cells Tissues Organs. 2002;172(2):126-32.) or a pro-drug converting enzyme, such as HSV thymidine kinase, under the control of a cancer-specific promoter. The cancer-specific promoter may be based on the promoter of the oncogene that is being knocked down. In that way, it is possible to provide targeted delivery of the mirtron to the target cell type and limit expression to cancerous tissue, while at the same time, enhance targeted drug activity by introducing a pro-drug converting enzyme not found naturally in the body that converts a pro-drug into a cytotoxic drug only in the cancerous tissue.

TABLE 2
Potential targets for siRNA therapy in cancer

| Pathway | Target gene |
|---|---|
| Apoptosis | Bax |
| | Bcl-2 |
| Angiogenesis | Focal Adhesion Kinase (FAK) |
| Adhesion | Matrix metalloproteinase |
| Cell-cell communication | VEGF |
| Lipid metabolism | Fatty acid synthase |
| Transport | MDR |
| Signalling | H-Ras |
| | K-Ras |
| | PLK-1 |
| | TGF-beta |
| | STAT3 |
| | EGFR |
| | PKC-alpha |
| Viral, oncogenes, nuclear | Epstein-Barr virus |
| | HPV E6 |
| | BCR-Abl |
| | Telomerase |

(Devi GR. siRNA-based approaches in cancer therapy.
Cancer Gene Ther. 2006 Sep; 13(9): 819-29. Epub 2006 Jan 20. Review).

Reporter System for Mirtron Expression

The mirtron of the invention may be codelivered as an intron in a reporter system. Using a reporter gene it is possible to trace the expression of the miRNA therapeutic temporally and spatially within the target tissues ex vivo with devices, such as the IVIS system® (Xenogen Corporation), rather than having to perform a biopsy, which is both expensive and time-consuming. The production of the mature miRNA species should be tightly correlated to the expression of the reporter gene. The correlation is due to the fact that both mirtron and reporter mRNA are driven by a single transcription unit and that both mirtron generation and reporter gene expression are splicing- and mutually-dependent. For any gene that can be targeted for knockdown in any disease it would be possible to track the expression profile of the RNAi therapeutic, thereby informing the healthcare provider of the progress and efficiency of the therapeutic and if it is necessary to deliver a repeated administration. Examples of reporter genes that can be used include Firefly luciferase and eGFP.

Therefore, the invention provides a vector comprising (i) a gene capable of expressing a mirtron and (ii) a reporter gene, wherein (i) and (ii) are under the control of the same promoter. The vector is suitable for use in monitoring the delivery and/or expression of the mirtron within the target mammalian tissue. The invention therefore provides said vector for use in monitoring the delivery and/or expression of the mirtron in the target mammalian tissue. The method of monitoring the delivery and/or expression of the mirtron comprises delivering said vector to a subject and monitoring the expression of the reporter gene. The reporter gene may be any suitable reporter gene and the method of monitoring the reporter gene may be any suitable method applicable to that reporter gene. Preferably the method of monitoring the expression of the reporter gene is non-invasive.

Polynucleotides

The mirtron of the invention comprises a nucleic acid sequence that has the ability to enter into the RNAi pathway as a miRNA bypassing Drosha. The mirtron of the invention may be delivered on its own, i.e. without further nucleotide sequence. Preferably however, the mirtron is delivered in a polynucleotide construct. Preferably, the mirtron takes the place of a natural intron within the sequence of a gene in the construct. That gene may for example be a gene that is being used to “replace” an endogenous defective gene in gene therapy. The invention therefore also relates to polynucleotide constructs comprising nucleic acid sequences which express a mirtron sequence of the invention. The constructs may comprise one or more nucleic acid sequences that encode one or polypeptides.

The terms “nucleic acid molecule” and “polynucleotide” are used interchangeably herein and refer to a polymeric form of nucleotides of any length, either deoxyribonucleotides or ribonucleotides, or analogs thereof Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form.

A nucleic acid sequence which “encodes” a polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

In one embodiment, a polynucleotide of the invention comprises a sequence corresponding to any of the mammalian mirtron sequences as defined herein or a variant of one of these specific sequences. For example, a variant may be a substitution, deletion or addition variant of any of these nucleic acid sequences. A variant may comprise 1, 2, 3, 4, 5, up to 10, up to 20, up to 30, up to 40, up to 50, up to 75 or more nucleic acid substitutions and/or deletions from the sequences provided. If the mirtron sequence is being provided in place of a natural intron within a gene in a construct, the sequence must be capable of being spliced. The sequence must be capable of entering the RNAi pathway and targeting the desired sequence for downregulation.

Suitable variants may be at least 70% homologous to polynucleotide sequence defined herein, preferably at least 80 or 90% and more preferably at least 95%, 97% or 99% homologous thereto. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPS containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologues typically hybridize with the relevant polynucleotide at a level significantly above background. The signal level generated by the interaction between the homologue and the polynucleotide is typically at least 10 fold, preferably at least 100 fold, as intense as “background hybridisation”. The intensity of interaction may be measured, for example, by radiolabelling the probe, e.g. with ³²P. Selective hybridisation is typically achieved using conditions of medium to high stringency, (for example, 0.03M sodium chloride and 0.003M sodium citrate at from about 50° C. to about 60° C.

Stringent hybridization conditions can include 50% formamide, 5× Denhardt's Solution, 5×SSC, 0.1% SDS and 100 μg/m1 denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

Polynucleotides of the invention can be synthesised according to methods well known in the art, as described by way of example in Sambrook et al (1989, Molecular Cloning —a laboratory manual; Cold Spring Harbor Press).

Vectors

The nucleic acid molecules of the present invention may be provided in the form of an expression cassette which includes control sequences operably linked to the inserted sequence, thus allowing for expression of the polynucleotide containing the mirtron of the invention in vivo in a targeted subject species. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of the polynucleotide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

Thus, a polynucleotide of the invention may be provided by delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extrachromosomal genetic element. The vectors may contain one or more selectable marker genes, for example an ampicillin resistence gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polynucleotide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

Promoters and other expression regulation signals may be selected to be compatible with the host cell for which expression is designed. For example, mammalian promoters, such as β-actin promoters, may be used. Tissue-specific promoters are especially preferred. Mammalian promoters include the metallothionein promoter which can be induced in response to heavy metals such as cadmium.

In one embodiment a viral promoter is used to drive expression from the polynucleotide. Typical viral promoters for mammalian cell expression include the SV40 large T antigen promoter, adenovirus promoters, the Moloney murine leukaemia virus long terminal repeat (MMLV LTR), the mouse mammary tumor virus LTR promoter, the rous sarcoma virus (RSV) LTR promoter, the SV40 early promoter, the human cytomegalovirus (CMV) IE promoter, adenovirus, including the adenovirus major late promoter (Ad MLP), HSV promoters (such as the HSV IE promoters), or HPV promoters, particularly the HPV upstream regulatory region (URR). All these promoters are readily available in the art.

In one embodiment, the promoter is a Cytomegalovirus (CMV) promoter. A preferred promoter element is the CMV immediate early (IE) promoter devoid of intron A, but including exon 1. Thus the expression from the polynucleotide may be under the control of hCMV IE early promoter. Expression vectors using the hCMV immediate early promoter include for example, pWRG7128, and pBC12/CMV and pJW4303. A hCMV immediate early promoter sequence can be obtained using known methods. A native hCMV immediate early promoter can be isolated directly from a sample of the virus, using standard techniques. U.S. Pat. No. 5,385,839, for example, describes the cloning of a hCMV promoter region. The sequence of a hCMV immediate early promoter is available at Genbank #M60321 (hCMV Towne strain) and X17403 (hCMV Ad169 strain). A native sequence could therefore be isolated by PCR using PCR primers based on the known sequence. See e.g Sambrook et al, supra, for a description of techniques used to obtain and isolate DNA. A suitable hCMV promoter sequence could also be isolated from an existing plasmid vector. Promoter sequences can also be produced synthetically.

A polynucleotide, expression cassette or vector of the invention may comprise an untranslated leader sequence. In general the untranslated leader sequence has a length of from about 10 to about 200 nucleotides, for example from about 15 to 150 nucleotides, preferably 15 to about 130 nucleotides. Leader sequences comprising, for example, 15, 50, 75 or 100 nucleotides may be used. Generally a functional untranslated leader sequence is one which is able to provide a translational start site for expression of a coding sequence in operable linkage with the leader sequence.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence.

Expression systems often include transcriptional modulator elements, referred to as “enhancers”. Enhancers are broadly defined as a cis-acting agent, which when operably linked to a promoter/gene sequence, will increase transcription of that gene sequence. Enhancers can function from positions that are much further away from a sequence of interest than other expression control elements (e.g. promoters), and may operate when positioned in either orientation relative to the sequence of interest. Enhancers have been identified from a number of viral sources, including polyoma virus, BK virus, cytomegalovirus (CMV), adenovirus, simian virus 40 (SV40), Moloney sarcoma virus, bovine papilloma virus and Rous sarcoma virus. Examples of suitable enhancers include the SV40 early gene enhancer, the enhancer/promoter derived from the long terminal repeat (LTR) of the Rous Sarcoma Virus, and elements derived from human or murine CMV, for example, elements included in the CMV intron A sequence.

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus.

Nucleic acids encoding for polypeptides with useful therapeutic properties may be included in a polynucleotide, expression cassette or vector of the invention. Alternatively, such polypeptides may be provided separately, for example in a formulation comprising a molecule of the invention, or may be administered simultaneously, sequentially or separately with a composition of the invention.

Methods for gene delivery are known in the art. See, e.g., U.S. Pat. Nos. 5,399,346, 5,580,859 and 5,589,466. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells which have been removed from a subject. In this latter case, cells containing the nucleic acid molecule of interest are re-introduced into the subject.

Each of these delivery techniques requires efficient expression of the nucleic acid in the transfected cell, to provide a sufficient amount of the therapeutic product. Several factors are known to affect the levels of expression obtained, including transfection efficiency, and the efficiency with which the gene or sequence of interest is transcribed and the mRNA translated.

The vectors and expression cassettes of the present invention may be administered directly as “a naked nucleic acid construct”, preferably further comprising flanking sequences homologous to the host cell genome. As used herein, the term “naked DNA” refers to a vector such as a plasmid comprising a polynucleotide of the present invention together with a short promoter region to control its production. It is called “naked” DNA because the vectors are not carried in any delivery vehicle. When such a vector enters a host cell, such as a eukaryotic cell, the proteins it encodes are transcribed and translated within the cell.

The vector of the invention may thus be a plasmid vector, that is, an autonomously replicating, extrachromosomal circular or linear DNA molecule. The plasmid may include additional elements, such as an origin of replication, or selector genes. Such elements are known in the art and can be included using standard techniques. Numerous suitable expression plasmids are known in the art. For example, one suitable plasmid is pSG2. This plasmid was originally isolated from Streptomyces ghanaensis. The length of 13.8 kb, single restriction sites for HindIII, EcoRV and PvuII and the possibility of deleting non-essential regions of the plasmid make pSG2 a suitable basic replicon for vector development.

Alternatively, the vectors of the present invention may be introduced into suitable host cells using a variety of viral techniques which are known in the art, such as for example infection with recombinant viral vectors such as retroviruses, herpes simplex viruses and adenoviruses.

In one embodiment, the vector itself may be a recombinant viral vector. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors. In the case of viral vectors, administration of the polynucleotide is mediated by viral infection of a target cell.

A number of viral based systems have been developed for transfecting mammalian cells.

For example, a selected recombinant nucleic acid molecule can be inserted into a vector and packaged as retroviral particles using techniques known in the art. The recombinant virus can then be isolated and delivered to cells of the subject either in vivo or ex vivo. Retroviral vectors may be based upon the Moloney murine leukaemia virus (Mo-MLV). In a retroviral vector, one or more of the viral genes (gag, pol & env) are generally replaced with the gene of interest.

A number of adenovirus vectors are known. Adenovirus subgroup C serotypes 2 and 5 are commonly used as vectors. The wild type adenovirus genome is approximately 35 kb of which up to 30 kb can be replaced with foreign DNA. There are four early transcriptional units (E1, E2, E3 & E4), which have regulatory functions, & a late transcript, which codes for structural proteins. Adenovirus vectors may have the E1 and/or E3 gene inactivated. The missing gene(s) may then be supplied in trans either by a helper virus, plasmid or integrated into a helper cell genome. Adenovirus vectors may use an E2a temperature sensitive mutant or an E4 deletion. Minimal adenovirus vectors may contain only the inverted terminal repeats (ITRs) & a packaging sequence around the transgene, all the necessary viral genes being provided in trans by a helper virus. Suitable adenoviral vectors thus include Ad5 vectors and simian adenovirus vectors.

Viral vectors may also be derived from the pox family of viruses, including vaccinia viruses and avian poxvirus such as fowlpox vaccines. For example, modified vaccinia virus Ankara (MVA) is a strain of vaccinia virus which does not replicate in most cell types, including normal human tissues. A recombinant MVA vector may therefore be used to deliver the polypeptide of the invention.

Addition types of virus such as adeno-associated virus (AAV) and herpes simplex virus (HSV) may also be used to develop suitable vector systems.

As an alternative to viral vectors, liposomal preparations can alternatively be used to deliver the nucleic acid molecules of the invention. Useful liposomal preparations include cationic (positively charged), anionic (negatively charged) and neutral preparations, with cationic liposomes particularly preferred. Cationic liposomes may mediate intracellular delivery of plasmid DNA and mRNA.

As another alternative to viral vector systems, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). Other particulate systems and polymers can also be used, for example, polymers such as polylysine, polyarginine, polyornithine, spermine, spermidine, as well as conjugates of these molecules.

In one embodiment, the vector may be a targeted vector, that is a vector whose ability to infect or transfect or transduce a cell or to be expressed in a host and/or target cell is restricted to certain cell types within the host subject, usually cells having a common or similar phenotype.

Preferably, a vector of the invention comprises a mirtron or a gene capable of expressing a mirtron as defined herein. The vector may comprise more than one mirtrons, for example 1, 2 or 3 or more mirtrons. These mirtrons may target the same gene or multiple genes. The mirtron sequences may be present in place of one or more natural introns within a gene sequence. The gene sequence may be the wild type gene sequence that is being used to “replace” a defective gene. The vector may comprise a reporter gene and/or one or more nucleic acid sequences that encode a useful polypeptide sequence for codelivery.

Pharmaceutical Compositions

Formulation of a composition comprising a molecule of the invention, such as a polynucleotide, expression cassette, or vector as described above, can be carried out using standard pharmaceutical formulation chemistries and methodologies all of which are readily available to the reasonably skilled artisan. For example, compositions containing one or more molecules of the invention can be combined with one or more pharmaceutically acceptable excipients or vehicles. Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e. g., sterile pyrogen-free water) prior to parenteral administration of the reconstituted composition. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono-or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Feigner et al. (1987) Proc. Natl. Acad. Sci. USA 84:7413-7416; Malone et al. (1989) Proc. Natl. Acad. Sci. USA 86:6077-6081; U.S. Pat. Nos 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules. The formulated compositions will include an amount of the molecule (e.g. vector) of interest which is sufficient to mount an immunological response. An appropriate effective amount can be readily determined by one of skill in the art. Such an amount will fall in a relatively broad range that can be determined through routine trials. The compositions may contain from about 0.1% to about 99.9% of the vector and can be administered directly to the subject or, alternatively, delivered ex vivo, to cells derived from the subject, using methods known to those skilled in the art.

Subject to be Treated

The subject to be treated may be any mammalian species, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs. The terms do not denote a particular age. Thus, both adult and newborn individuals are intended to be covered. The subject will preferably be a human, but may also be a domestic livestock, laboratory subject or pet animal.

Delivery Methods

Once formulated the compositions can be delivered to a subject in vivo using a variety of known routes and techniques. For example, a composition can be provided as an injectable solution, suspension or emulsion and administered via parenteral, subcutaneous, epidermal, intradermal, intramuscular, intraarterial, intraperitoneal, intravenous injection using a conventional needle and syringe, or using a liquid jet injection system. Compositions can also be administered topically to skin or mucosal tissue, such as nasally, intratracheally, intestinal, rectally or vaginally, or provided as a finely divided spray suitable for respiratory or pulmonary administration. Other modes of administration include oral administration, suppositories, and active or passive transdermal delivery techniques. Particularly in relation to the present invention, compositions may be administered directly.to the gastrointestinal tract. As explained above, the mirtron may be delivered as a construct within a suitable vector by any gene delivery system available for gene therapy, such as virus vectors, and plasmids in liposome formulation. Alternatively, the compositions can be administered ex vivo, for example delivery and reimplantation of transformed cells into a subject are known (e.g., dextran-mediated transfection, calcium phosphate precipitation, electroporation, and direct microinjection into nuclei).

Delivery Regimes

The compositions are administered to a subject in an amount that is compatible with the dosage formulation and that will be prophylactically and/or therapeutically effective. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed.

As used herein, the term “prophylactically or therapeutically effective dose” means a dose in an amount sufficient to alleviate, reduce, cure or at least partially arrest symptoms and/or complications from a disease.

Prophylaxis or therapy can be accomplished by a single direct administration at a single time point or by multiple administrations, optionally at multiple time points. Administration can also be delivered to a single or to multiple sites. Those skilled in the art can adjust the dosage and concentration to suit the particular route of delivery. In one embodiment, a single dose is administered on a single occasion. In an alternative embodiment, a number of doses are administered to a subject on the same occasion but, for example, at different sites. In a further embodiment, multiple doses are administered on multiple occasions. Such multiple doses may be administered in batches, i.e. with multiple administrations at different sites on the same occasion, or may be administered individually, with one administration on each of multiple occasions (optionally at multiple sites). Any combination of such administration regimes may be used.

Different administrations may be performed on the same occasion, on the same day, one, two, three, four, five or six days apart, one, two, three, four or more weeks apart. Preferably, administrations are 1 to 5 weeks apart, more preferably 2 to 4 weeks apart, such as 2 weeks, 3 weeks or 4 weeks apart. The schedule and timing of such multiple administrations can be optimised for a particular composition or compositions by one of skill in the art by routine trials.

Dosages for administration will depend upon a number of factors including the nature of the composition, the route of administration and the schedule and timing of the administration regime. The dose will also vary according to the severity of the condition, age, and weight of the patient to be treated. A physician will be able to determine the required route of administration and dosage for any particular patient. Optimum dosages may vary depending on the relative potency of the nucleic acids, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10mg/kg of body weight, according to the potency of the nucleic acid, the age, weight and condition of the subject to be treated, the severity of the disease and the frequency and route of administration.

The invention is illustrated by the following Examples:

### EXAMPLE 1

Materials and Methods

Plasmid Construction:

Two neighbouring sections of eGFP from the eGFP-C1 vector (Clontech) which had a splice site sequence trait at their junction were PCR amplified using two primer sets. Subsequent digestions with NheI/XhoI and XhoI/HindIII respectively allowed sub-cloning back into the eGFP-C1 vector backbone. Bbs1 restriction sites were incorporated appropriately into primer sets giving the new construct an eGFP sequence with BbsI restriction sites between the splice-site like sequence: 5′-TCTTCAAG/GACGACGG. All subsequent synthetic introns were introduced as annealed double-stranded oligonucleotides with 5′-CAAG sense strand overhang and 5′-CGTC anti-sense strand overhang into Bbs 1-digested eGFP-Mirt plasmid.

eGFP 1 F′:
(SEQ ID NO: 65)
5′-AGATCCGCTAGCGCTACCGGTC-3′
eGFP 1 R′:
(SEQ ID NO: 66)
5′-GTCTACTCGAGAAGACTACTTGAAGAAGATGGTGCGC-3′
eGFP 2 F′:
(SEQ ID NO: 67)
5′-GTCTTCTCGAGAAGACTTGACGACGGCAACTACAAGA-3′
eGFP 2 R′:
(SEQ ID NO: 68)
5′-ATCGAAGCTTACTTGTACAGCTCGTCCAT-3′

Luciferase Targets:

Target sequences of mirtrons used in the luciferase assay were inserted in-frame with the Renilla Luciferase gene in the psiCheck2.2 dual-luciferase cassette (Promega) using Xho1 and Not 1 restriction sites.

Cell Transfection:

All experiments were performed in HEK-293 cells (ATCC, CRL-1573) cultured in DMEM supplemented with 10% FBS, 100U/ml penicillin and 100 μg/ml streptomycin at 37° C. and 5% CO₂. For luciferase assays, unless stated otherwise, mirtrons and targets were transfected in a 1:1 ratio using Lipofectamine 2000 at final concentrations of 500 ng/m1 per construct.

Luciferase Assays:

Cells were lysed at 48 hrs post-transfection and dual-luciferase readings obtained using the Promega Dual-Luciferase kit and Wallac-Victor 2 plate reader as per manufacturer's instructions. Ratios of renilla luciferase:firefly luciferase were obtained as a measure of gene-silencing and normalised to a non-specific control intron insert.

Fluorescence Assay:

Cells were lysed 48 hours post-transfection and protein content was determined using the Bradford method. 100 ng of protein was assayed for GFP fluorescence using a Wallac-Victor 2 plate reader as per manufacturer's instructions. Background fluorescence from un-transfected cells was subtracted. For the measurement of α-synuclein-mCherry, mCherry fluorescence was normalised to the NADPH-intron transfected cells.

Western Blot:

HEK-293 cells were lysed 48hrs post-transfection using 100 ul of RIPA buffer and protein content was determined using the Bradford method. 100 ng of protein was from each sample was used for SDS-PAGE and subsequent Western Blot using anti-eGFP (Molecular Probes, A-11122) and anti-cyclophilin B (Abeam, A3565) as primary antibodies.

Results

Development of a synthetic mirtron expression system and demonstration of gene silencing with miR-877, a mammalian mirtron

An eGFP plasmid was engineered to allow any synthetic intron to be inserted (FIG. 3A). Successful splicing results in eGFP fluorescence due to the production of a shortened, functional mRNA transcript for eGFP. To test the ability of mirtrons to work in mammalian cells, a target to the predicted mammalian mirtron miR-877 was fused to the renilla luciferase gene in a dual-luciferase cassette (FIG. 3B). The miR-877 sequence, together with splicing-deficient mutant sequences (FIG. 3C) and a non-hairpin forming NAD-intron control (derived from a short intron from the human sequence for NADPH dehydrogenase, similar in length to mirtrons), were inserted into the eGFP sequence and co-transfected with luciferase target into HEK-293 cells. Fluorescence imaging and reverse-transcription PCR revealed that only the control intron and miR-877 sequence were spliced out (FIG. 3D and E). Dual luciferase screening demonstrated that only miR-877 was able to knock-down the levels of miR-877 target tagged renilla luciferase to levels of 27% of the non-specific control intron.

The results demonstrate the usefulness of the synthetic mirtron expression system that we have developed in designing and testing potential mirtrons for splicing and gene silencing capabilities. Furthermore, the results provide the first demonstration of gene silencing with a mammalian mirtron.

Rational Design of Synthetic Mirtron against mRNA Targets

The first round of designs was based on a simple miRNA structure (miR-30) with full complementary between the two arms of the hairpin and an artificial stem loop designed with a branch point within (Table 3). In designs 1.1, 3.1, 3.2, 5.1 and 5.2 the antisense sequence was located in the 5′ arm of the hairpin loop. As all of the first round designs failed to produce good eGFP signal in HEK cells and failed to knockdown cyclophilin B as determined by Western blotting, we believe that these designs were ultimately hampered by their inability to splice properly. The 5′ arms of the construct had a matching 5′ consensus sequence of GUxAGU. The branch points were designed to match the U2 branch point recognition site incorporated into a pre-validated miRNA hairpin of miR-30. However the polypyrimidine tracts, located within the 3′ arms, were not pyrimidine-rich enough (<70% pyrimidine in first 20nts) and the full complementarity of the two arms, something not found in natural mirtrons, could have hindered the recognition of the polypyrimidine tract by the splicesosome. These two reasons, we believe, were the causes of ineffective splicing and the second round of designs addressed these problems by reducing the complementarity of the two arms by introducing mismatches and mimicking the natural bulges of human miR-877 and selecting silencing sequences that are more pyrimidine-rich (>70%).

The second set of designs had 2 constructs that spliced out better than mirt 1.1, but no knockdown of cyclophilin was observed. We believe the lack of splicing for two of the designs was due to the fact that the polypyrimidine tract may have had too many Cs and too many pyrimidines, which may result in the 3′ arm binding too strongly to the 5′ arm, thus obscuring recognition of the polypyrimidine tract. For the 2 sequences that spliced, we did not detect any significant knockdown using Western Blots and since these RNA sequences have not been validated as siRNA sequences, we thought that the miRNAs may have been produced, but these are not effective at knocking down the targets. Given these possibilities, we decided to use a validated siRNA sequence against cyclophilin B which has been shown to knock down cyclophilin in HEK cells when delivered as a double strand RNA. Also to eliminate the possiblity that the target mRNA may be problematic, we also designed mirtrons that can knock down luciferase as an alternative.

The third round of designs resulted in luciferase-targeting splicing-competent structures, but again, no target knock down was observed by luciferase assay. We used the miR-1226 hairpin instead of miR-877 for mirt 21 to test if changing the hairpin will make a difference (FIG. 4A). The hypothesis in doing this was because mir-1226 is predicted to use its 3′ arm as the anti-sense strand and the anti-sense of the control siRNA targeting cyclophilin is pyrimidine rich and would potentially fit in as the 3′ arm of this mirtron with ease. Mirt 21B is essentially the same design with a miR-877 hairpin loop. As there is no discernable branch point in miR-1226 hairpin, we engineered in one while retaining the essential structure and labelled it Mirt 21C. Mirt 21 and Mirt 21 B failed to splice while Mirt 21 C both spliced (FIG. 4B) and knocked down cyclophilin B, making this the first example of a synthetically designed mirtron capable of performing RNAi. The result was first shown by Western blotting of cyclophilin B (FIG. 4C), then verified with artificial targets inserted downstream of a luciferase gene and assayed for knockdown (FIG. 4D). A quantitative RT-PCR was also performed to ascertain the results (FIG. 4E). We are now in the process of investigating if an optimized branch point may be able to improve splicing efficiency (mirt 21OBP).

We have furthered this data by designing synthetic mirtrons targeting the Parkinson's Disease linked alpha-synuclein gene. To date three mutations and a gene triplication of the alpha-synuclein gene have been linked to Parkinson's Disease, and aggregates of this protein are implicated in the disease pathology making this an attractive target for mirtron targeting. Of the 6 mirtrons designed to target the mRNA of alpha-synuclein, 3 showed splicing ability (Table 3). When tested against dual luciferase synthetic targets, one of these mirtrons demonstrated a silencing effect of ˜25% relative to the NAD control intron (FIG. 5A). This degree of silencing was mirrored by the complementary snRNA construct in which the mirtron sequence was expressed off a separate U6 promoter rather than being expressed in the context of an intron as a mirtron. When the successful mirtron, pMirt A-syn 1, was directed against a full-length alpha-synuclein mRNA transcript with mCherry fluorescent tag, a reduction in mCherry fluorescence of ˜25% was seen relative to the NAD intron (FIG. 5B). The result demonstrates a second synthetic mirtron capable of reducing the expression of a full-length target gene.

TABLE 3
Mirtron inserts

| | | | | | Target |
|---|---|---|---|---|---|
| | | Tar- | | Splic- | Knock |
| Name | Sequence | get | Purpose | ing? | Down? |
| NADintron | GTATGTAGTAGAATTCTGTCAATCTTTTTGGTTGTCTCAGATTTTA | NA | Control | Yes | NA |
| (SEQ ID NO: 69) | ATTTTATTAGCAGCATGAGATTGACTCTTTCATAATCTACTTAAG | | intron from | | |
| | | | human NADH | | |
| | | | dehydrogenase | | |
| miR877 | GTAGAGGAGATGGCGCAGGGGACACGGGCAAAGACTTGGGGGTT | ??? | Natural human | Yes | Yes |
| (SEQ ID NO: 70) | CCTGGGACCCTCAGACGTGTGTCCTCTTCTCCCTCCTCCCAG | | miR-877 | | |
| | | | sequence | | |
| miR877US | AGTGGGGAGATGGCGCAGGGGACACGGGCAAAGACTTGGGGGT | ??? | miR-877 with | No | No |
| (SEQ ID NO: 71) | TCCTGGGACCCTCAGACGTGTGTCCTCTTCTCCCTCCTCCCAG | | 5′ splice | | |
| | | | site mutation | | |
| | | | to prevent | | |
| | | | splicing | | |
| miR877BP | GTAGAGGAGATGGCGCAGGGGACACGGGCAAAGACTTGGGGGTT | ??? | miR-877 with | No | No |
| (SEQ ID NO: 72) | CCTGGGACCCTCCGGCGTGTGTCCTCTTCTCCCTCCTCCCAG | | branch point | | |
| | | | mutation to | | |
| | | | prevent | | |
| | | | splicing | | |
| miR1226 | GTGAGGGCATGCAGGCCTGGATGGGGCAGCTGGGATGGTCCAAA | ??? | Natural human | Yes | TBD |
| (SEQ ID NO: 73) | AGGGTGGCCTCACCAGCCCTGTGTTCCCTAG | | miR-1226 | | |
| | | | sequence | | |
| miR1226US | ACGAGGGCATGCAGGCCTGGATGGGGCAGCTGGGATGGTCCAAA | ??? | miR-1226 with | No | TBD |
| (SEQ ID NO: 74) | AGGGTGGCCTCACCAGCCCTGTGTTCCCTAG | | 5′ splice | | |
| | | | site mutation | | |
| | | | to prevent | | |
| | | | splicing | | |
| miR1226DUS | ACGAGGGCATGCAGGCCTGGATGGGGCAGCTGGGATGGTCCAAA | ??? | miR-1226 | No | TBD |
| (SEQ ID NO: 75) | AGGGTGGCCTCACCAGCCCTGTGTTCCCTGA | | with 5′ and 3′ | | |
| | | | splice site | | |
| | | | mutations | | |
| | | | to prevent | | |
| | | | splicing | | |
| miR1226BP | GTGAGGGCATGCAGGCCTGGATGGGGCAGCTGGGATGGTCCGGG | ??? | miR-1226 with | Yes | TBD |
| (SEQ ID NO: 76) | GGGGTGGCCTCACCAGCCCTGTGTTCCCTAG | | branch point | | |
| | | | mutation to | | |
| | | | prevent | | |
| | | | splicing | | |
| miR1224 | GTGAGGACTGGGGAGGTGGAGGGTAGCATCATTAGAGCCAGAGC | | Natural mouse | Yes | Yes |
| (SEQ ID NO: 77) | TCTGTCTCAGCTCCCTCTCCCCCCACCTCTTCTCTCCTCAG | | miR-1224 | | |
| | | | sequence | | |
| miR1224US | GCGAGGACTGGGGAGGTGGAGGGTAGCATCATTAGAGCCAGAGC | | miR-1224 | No | No |
| (SEQ ID NO: 78) | TCTGTCTCAGCTCCCTCTCCCCCCACCTCTTCTCTCCTCGG | | sequence with | | |
| | | | 5′ and 3′ | | |
| | | | splice site | | |
| | | | mutations | | |
| | | | to prevent | | |
| | | | splicing | | |

First round of synthetic constructs

| | | | | |
|---|---|---|---|---|
| mirt1.1 | GTGGGTTTTTGGAACAGTCTTTCCTTTGTTCTGGTCTGACCCATGG | cycB | Weak | No |
| (SEQ ID NO: 79) | GAAAGACTGTTCCAAAAATTCAG | | | |
| mirt3.1 | GTGGGTCAAAATACACCTTGACGGTGTTCTGGTCTGACCCATCGT | cycB | No | No |
| (SEQ ID NO: 80) | CAAGGTGTATTTTGACCCAG | | | |
| mirt3.2 | GTGGGAATTTGCTGTTTTTGTAGCTGTTCTGGTCTGACCCAGCTAC | cycB | No | No |
| (SEQ ID NO: 81) | AAAAACAGCAAATTCCCAG | | | |
| mirt5.1 | GTGGGGAAGAACTGGGAGCCCGTGTTCTGGTCTGACCCATGGCTC | cycB | No | No |
| (SEQ ID NO: 82) | CCAGTTCTTCTCCACCAG | | | |
| mirt5.2 | GTGGGTCAGTTTGAAGTTCTCTGTTCTGGTCTGACCCAGGGAACTT | cycB | No | No |
| (SEQ ID NO: 83) | CAAACTGATCCACCAG | | | |

Second round of synthetic constructs

| | | | | |
|---|---|---|---|---|
| mirt11 | GTAGGTCAAAATACACCTTG [symbol] ACACGGGCAAAGACTTGGGGGTTC | cycB | Yes | No |
| (SEQ ID NO: 84) | CTGGGACCCTCAGACGTGT [symbol] TCCAAGCTTATTTTCCTCCAG | | | |
| mirt12 | GTGGATCATGAAGTCCTTGA [symbol] ACACGGGCAAAGACTTGGGGGTTC | cycB | No | No |
| (SEQ ID NO: 85) | CTGGGACCCTCAGACGTGT [symbol] TCTCAAGTCCTTCTTCCCCAG | | | |
| mirt13 | GTGATGAAGAACTGGGAGCC [symbol] ACACGGGCAAAGACTTGGGGGTT | cycB | No | No |
| (SEQ ID NO: 86) | CCTGGGACCCTCAGACGTGT [symbol] TCGGCTCCCTTCTTCTCCCAG | | | |
| mirt14 | GTCAGTTTGAAGTTCTCATC [symbol] ACACGGGCAAAGACTTGGGGGTTC | cycB | Yes | No |
| (SEQ ID NO: 87) | CTGGGACCCTCAGACGTGT [symbol] TCGATGAGAACTTCCCACTCCAG | | | |

Third round of synthetic constructs

| | | | | |
|---|---|---|---|---|
| mirtFL1 | GTGAGATGTCACGAATGTGTGACACGGGCAAAGACTTGGGGGTTC | Luc | Yes | No |
| (SEQ ID NO: 88) | CTGGGACCCTCAGACGTGTGTCACACCTCCGACATCTCCAG | | | |
| mirtFL2 | GTGAGAAATGCCCATGCTGTGACACGGGCAAAGACTTGGGGGTTC | Luc | Yes | No |
| (SEQ ID NO: 89) | CTGGGACCCTCAGACGTGTGTCACAGTACAAATTTCTCCAG | | | |
| mirt21 | GTGAGGACAGACTGTCCCAAGAAAGGCAGCTGGGATGGTCCAAA | cycB | No | No |
| (SEQ ID NO: 90) | AGGGTGGCCTTTTTGGAACAGTCTTTCCTAG | | | |
| mirt21B | GTGAGGACAGACTGTCCCAAGAAAGGACACGGGCAAAGACTTGG | cycB | No | No |
| (SEQ ID NO: 91) | GGGTTCCTGGGACCCTCAGACGTGTGTCCTTTTTGGAACAGTCTTT | | | |
| | CCTAG | | | |
| mirt21C | GTGAGGACAGACTGTCCCAAGAAAGGCAGCTGGGATGGTCC [symbol] GA | cycB | Yes | Yes |
| (SEQ ID NO: 92) | CGGGTGGCCTTTTTGGAACAGTCTTTCCTAG | | | |
| mirt21OBP | GTGAGGACAGACTGTCCCAAGAAAGGCAGCTGGGATGGTCCTGA | cycB | Yes | TBD |
| (SEQ ID NO: 93) | CGGGTGGCCTTTTTGGAACAGTCTTTCCTAG | | | |

Alpha-synuclein synthetics

| | | | |
|---|---|---|---|
| pMirt A-syn 1 | gtggagcctacatagagaacaggtagcatcattagagccagagctct | Yes | Yes |
| (SEQ ID NO: 94) | gtctcagctccttctccccttctctatgttcgctccag | | |
| pMirt A-syn 2 | gugacagaggcugaggagacacaaggcaggagggaugguccagacgu | No | No |
| (SEQ ID NO: 95) | ccugccuuggucuucucagccacuguag | | |
| pMirt A-syn 3 | gugcgugagugagaggaccgggguagcaucauuagagccagagcucu | No | No |
| (SEQ ID NO: 96) | gucucagcucccuccccuuggucuucucagccauguag | | |
| pMirt A-syn 4 | gugggcaaaagcagccggaagagaggcaggagggaugguccagacgu | Weak | No |
| (SEQ ID NO: 97) | ccugccucuuuccugcugcuucugccag | | |
| pMirt A-syn 5 | gugcggccuuagcccggaggagguagcaucauuagagccagagcucu | Weak | No |
| (SEQ ID NO: 98) | gucucagcucccuccccucuucugggcuacugcuguag | | |
| pMirt A-syn 6 | gugggcaccaguagcgcagaaagaggcaggagggauggguccagacg | Weak | No |
| (SEQ ID NO: 99) | uccugccucuucuggcuacugcugucag | | |

TABLE 4
Mirtron alignments

| Mirtron | Structure |
|---|---|
| 877 | 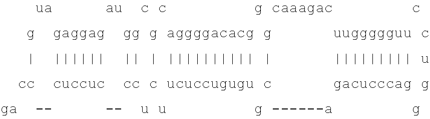 |
| 1226 | 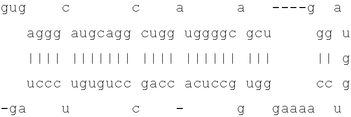 |
| 1224 | 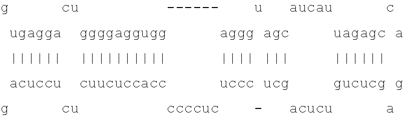 |
| 1.1 | 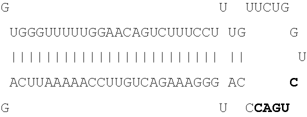 |
| 3.1 | 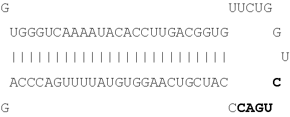 |
| 3.2 | 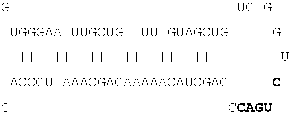 |
| 5.1 | 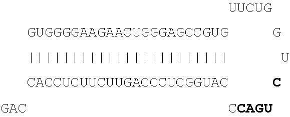 |
| 5.2 | 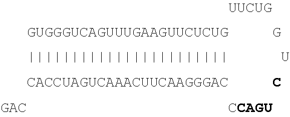 |
| 11 | 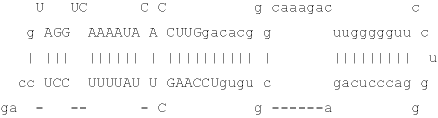 |
| 12 | 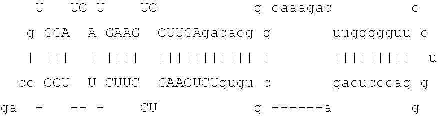 |
| 13 | 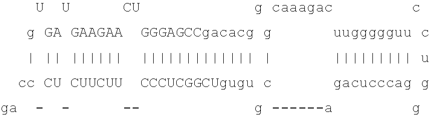 |
| 14 | 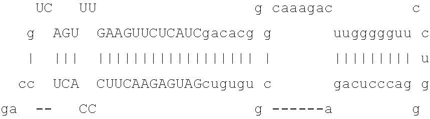 |
| FL 1 | 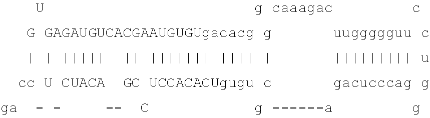 |
| FL 2 | 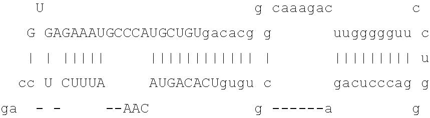 |
| 21 | 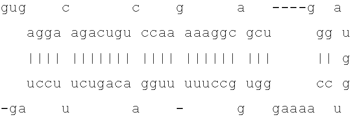 |
| 21B | 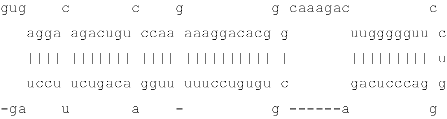 |
| 21C | 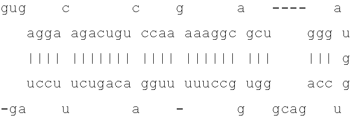 |
| 21OBP | 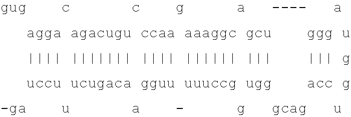 |
| ASyn 1 | 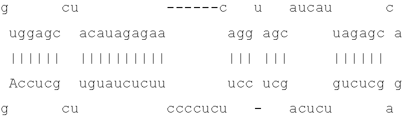 |
| ASyn 2 | 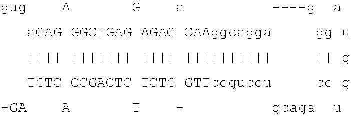 |
| ASyn 3 | 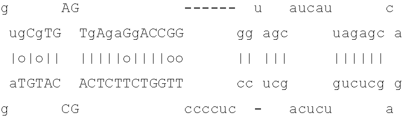 |
| ASyn 4 | 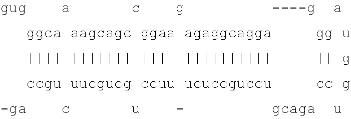 |
| ASyn 5 | 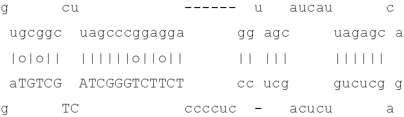 |
| ASyn 6 | 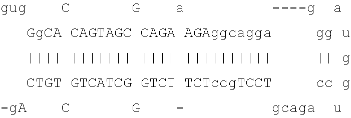 |

### EXAMPLE 2

Coupling Gene Replacement to Mirtron-Mediated Knockdown

Type I myotonic dystrophy (DM1) is a disease that affects approximately 1 in 8000 individuals caused by an autosomal dominant unstable CTG expansion in the 3′-untranslated (3′UTR) region of the myotonic dystrophy protein kinase (DMPK) gene. Several mechanisms contribute to the symptoms observed in patients. The CTG expansion results in nuclear retention of DMPK mRNA which leads to reduced production of the DMPK protein. Evidence for normal DMPK expression being essential for normal muscle function comes from DMPK knockout mice models which develop myopathy and cardiac abnormalities. The CUG hairpin formed also sequesters CUG-binding proteins which result in abnormal splicing and is also causative of some symptoms of the disease (Kaliman P et al., Myotonic dystrophy protein kinase (DMPK) and its role in the pathogenesis of myotonic dystrophy 1. Cell Signal. 2008 Nov;20(11):1935-41. Epub 2008 May 18. Review).

Hence, knockdown of the mutant DMPK gene must be achieved without drastic reduction of the DMPK protein. However, allele-specific silencing of DMPK has proven to be elusive as it is difficult to differentiate the number of CTG repeats, although CAG repeat antisense oligonucleotides appear promising as a therapy (Wheeler T M et al., Reversal of RNA dominance by displacement of protein sequestered on triplet repeat RNA. Science. 2009 Jul 17;325(5938):336-9). In our opinion, the best method for maintaining DMPK expression is to use a synthetic mirtron to knockdown mutant endogenous gene expression while simultaneously expressing a codon-replaced RNAi-resistant DMPK gene.

In order to identify synthetic mirtrons that can knockdown the DMPK gene, we utilized a mirtron target identification algorithm which uses the rules for mirtron design described herein and designed synthetic mirtrons which incorporated natural mirtron hairpins (FIG. 7). 15 potential mirtron target sequences were identified and 6 were chosen and cloned into the pEGFP-Mirt vector as described in Example 1. GFP expression and the resultant fluorescence will imply the relative efficacy of splicing of the respective mirtrons while knockdown can be assayed with a dual-luciferase construct incorporating the target sequence in the 3′UTR. In this particular set of experiments, the target luciferase vector incorporated target sequences of DMPK Mirt 1, 4, 5 and 6 in series to detect knockdown efficiency of the mirtrons.

FIG. 8A shows the effective knockdown of the luciferase reporter by the 4 different mirtrons. As controls, a target only transfection and an intron from human NADPH were used. Two of the mirtrons designed to incorporate the miR-1224 hairpin loop appears to be spliced as well as the NADPH intron based on the eGFP fluorescence (FIG. 8A and B), which suggests to us that the miR-1224 hairpin loop is more conducive for splicing as compared to our previous designs based on the miR-877 and miR-1226 hairpin loops (FIGS. 7 and 8C). Furthermore, it appears that our design algorithm is successfully picking up target sequences that allow mirtrons designed against them to conform to splicing requirements, namely the polypyrimidine-rich 3′ arm and the 5′ splice site.

One of our mirtrons appears to knock down the target sequence by over 80%, which demonstrates the capability of synthetic mirtrons to effect therapeutic levels of knock down. In order to demonstrate that the mirtron is dependent on splicing, we mutated the terminal guanosine nucleotide in the 5′ splice site to an adenosine, which should not affect silencing efficacy but would prevent splicing. As expected, the mutation abrogates eGFP splicing and target knockdown (FIG. 8D), thus demonstrating that the knockdown by the synthetic mirtron is dependent of splicing. More notably, the shRNA equivalent of the mirtron appears to be weaker at knocking down the target than the mirtron, which suggests that optimal design of antisense species derived from mirtrons may be different from that of shRNA.

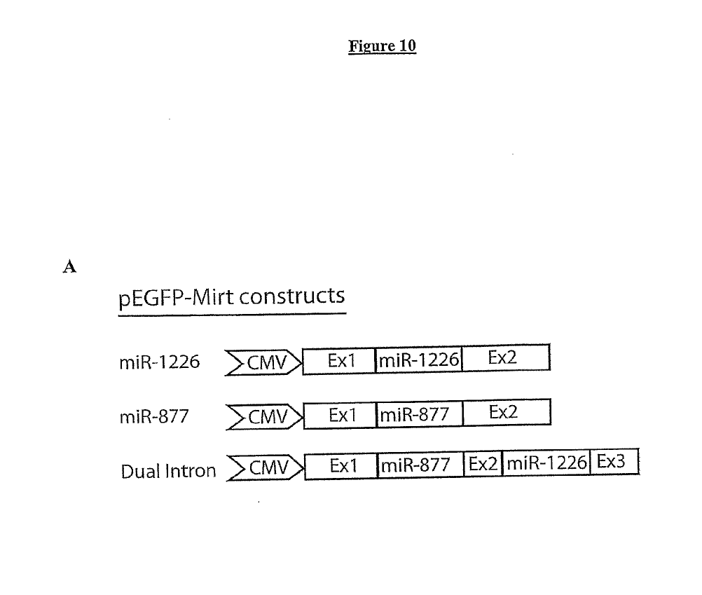

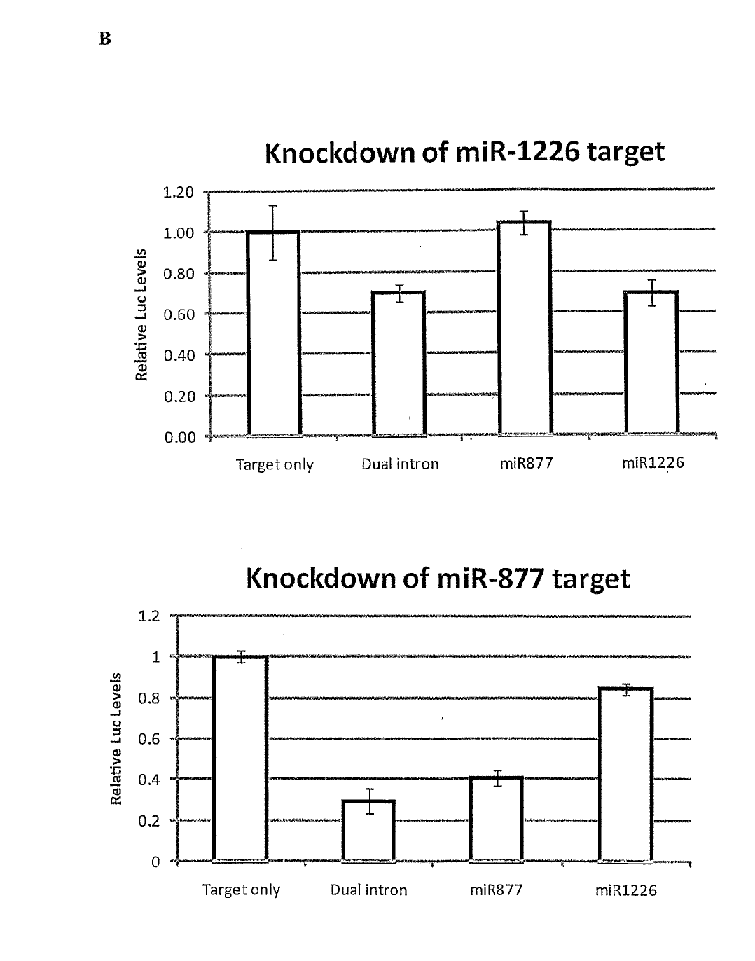

Lastly, we investigated if a codon-replaced RNAi-resistant version of the target (FIG. 9A) can be knocked down by DMPK Mirt 5. We also wanted to investigate the potential off-target effect of the mirtron, given that the 5′ splice site consensus overlaps significantly with the seed region of the antisense strand, potentially affecting transcripts that are targeted by natural mirtrons. We employed a miR-877 target, the target of a natural mirtron with a similar seed region, to ascertain the off-target potential of DMPK Mirt 5. Based on the knockdown data in FIG. 9B, there appears to be no knockdown of the RNAi-resistant target, suggesting that the mirtron can be used for a gene-knockdown-and-replacement strategy. Similarly, there appears to be no significant knockdown of the miR-877 target, suggesting that despite the similarity in seed region, synthetic mirtrons with the consensus 5′ splice site sequence in its seed region do not appear to possess significant off-targeting effects.

### EXAMPLE 3

Directed evolution of active anti-sense species into mirtrons

Sequences extraneous to the active anti-sense sequence, including the hairpin loop and sense strands, can have a major contribution to the silencing capabilities of RNAi effecters. Research has previously shown that the rational optimization of the hairpin sequence has enabled engineering of highly effective shRNA structures (Zhou H, Xia XG, Xu Z. An RNA polymerase II construct synthesizes short-hairpin RNA with a quantitative indicator and mediates highly efficient RNAi. Nucleic Acids Res. 2005 Apr. 1;33(6):e62.). Splicing and silencing activity of synthetic mirtron sequences targeting cyclophilin B and alpha-synuclein (Example 1) additionally shows dependence on sequences extraneous to the active anti-sense sequence. A difference in as little as one nucleotide can be sufficient to eliminate or improve activity of the mirtron. In order to incorporate highly potent and previously validated anti-sense species targeting genes of interest into mirtron sequences, we have selected directed evolution to optimize extraneous sequences to the anti-sense sequence to both a) improve splicing b) increase knockdown. The following experiments are geared towards this aim and have been performed in cell culture in HEK cells as a proof-of-principle.

1) Identify active mirtron sequences capable of knocking down cyclophilin

Example 1 identified a synthetic mirtron sequence capable of reducing cyclophillin B expression. A control shRNA which theoretically carries the same anti-sense species is able to reduce target expression by >80% whereas the mirtron variant can only reduce expression by ˜25%. Following several design iterations, splicing of the mirtron was improved with little improvement in silencing ability. To see whether this design of synthetic mirtron could be improved further with regards to both splicing and silencing efficiency, this mirtron, CycD2, was selected as the parent backbone for the randomised design process.

2) Incorporation of designs into mirtrons including randomised nucleotides in extraneous regions to anti-sense sequence, and generation of mirtron library

Within the CycD2 backbone, three nucleotides outside of the anti-sense strand were left randomised such that 3! permutations of the same parent construct could be potentially synthesised. Random nucleotide positions were selected on the hypothesis that they would improve the open up the branch point (1 position) or increase the poly-pyrimidine tract length (2 positions). Two additional nucleotides were made random within the anti-sense strand on the hypothesis that it would improve the polypyrimidine tract. Following incorporation into the pEGFP-Mirt vector, a randomised library containing, in theory, 5! permutations of the parent construct was generated. Optimized ligation and transformation reagents were used to ensure the largest possible plasmid library of possible mirtron permutations to maximize screening efficacy.

3) Screening of Library Constructs

Upon transfection of the plasmid into HEK cells, we are able to assess two critical aspects of mirtron biogenesis in a simple manner. If the mirtron is effectively spliced out, eGFP protein will be produced and the cell will fluoresce. If a miRNA species is successfully processed by DICER, a co-transfected dual luciferase target to this mirtron will be knocked down. Both aspects can be quantified using a plate reader.

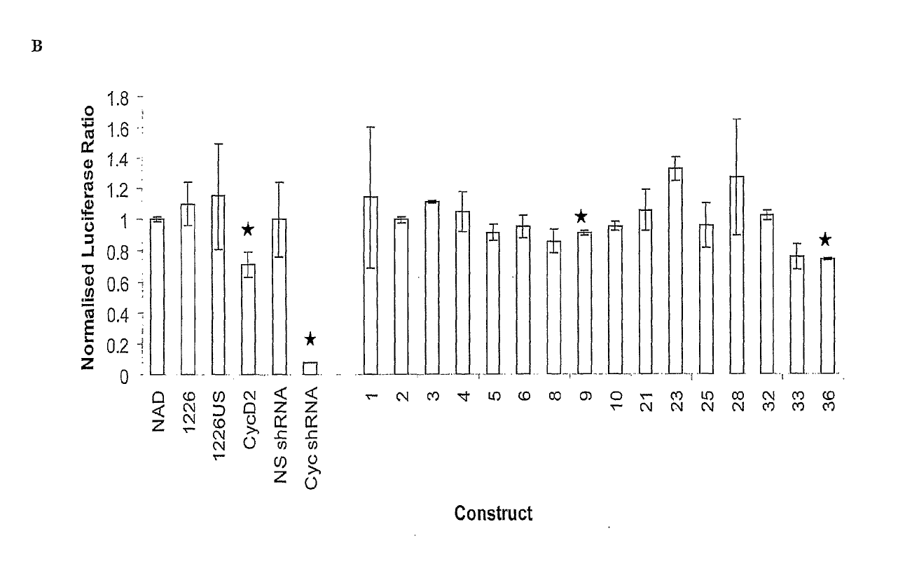

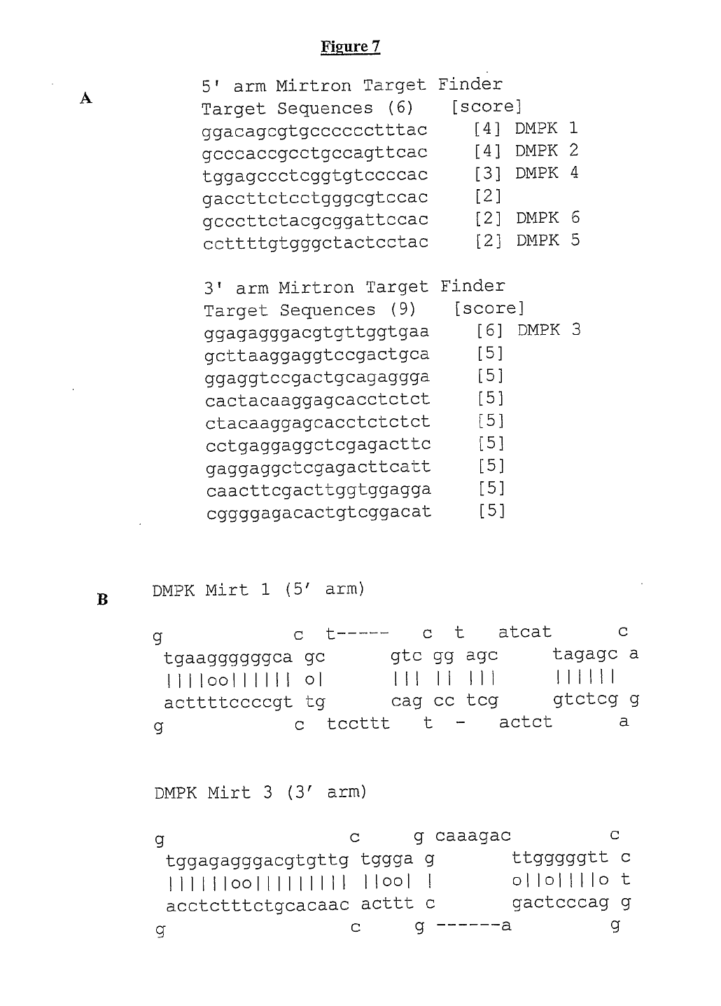

FIG. 6A demonstrates that the random library produces much variation in splicing efficiency with some constructs showing comparable splicing to that of the control NAD intron. The result confirms that variation in the mirtron sequence at individual nucleotides can alter the activity of synthetic mirtrons. FIG. 6B demonstrates that of the sequences which were spliced out, variation was seen in the ability to silence. Whereas the parent mirtron, CycD2, was able to reduce expression of the target by 25%, several of the randomly designed variants showed no ability to silence suggesting their individual sequences were not suitable as RNAi effecters against this target. However one randomly designed mirtron, Cyc36, showed a significant silencing effect by 22%. Although silencing was not as large as that with CycD2, the splicing appeared much stronger when comparing GFP levels. Taken together the result shows that a synthetic mirtron can be randomly evolved and this may be a suitable approach for evolving previously validated RNAi effectors into synthetic mirtrons for future applications.

1. A method of modifying the expression of a target gene in a mammalian cell to prevent or treat a disease, comprising administering a mirtron or a gene capable of expressing a mirtron, sequence of the mirtron comprising:
(i) a 5′ splice site;
(ii) a 3′ splice site;
(iii) a branch-point recognition sequence;
(iv) a 3′ polypyrimidine tract greater than 15 nucleotides in length; and
(v) an antisense sequence that is at least partially complementary to a sequence in the target gene.

2. The method according to claim 1, wherein the mirtron sequence is 45 to 200 nucleotides in length.

3. The method according to claim 1, for treating a genetic disease by reducing or eliminating the expression of a target defective gene.

4. The method according to claim 3, wherein the target gene comprises a dominant gain-of-function mutation.

5. The method according to claim 3, wherein the target gene and/or disease to be treated is selected from the genes and/or diseases defined in Table 1 or 2.

6. The method according to claim 1, wherein the mirtron or gene is administered in combination with a modified version of the target gene that is not recognised by the mirtron in replacement gene therapy.

7. The method according to claim 1, comprising administering a vector comprising the mirtron or gene capable of expressing a mirtron.

8. The method according to claim 7, wherein the vector further comprises:
(i) a reporter gene; and/or
(ii) a tissue-specific promoter; and/or
(iii)a modified version of the target gene that is not recognised by the mirtron for use in replacing the target gene.

9. A method of monitoring the delivery and/or expression of a mirtron in a target mammalian tissue, comprising administeringa vector comprising (i) a gene capable of expressing a mirtron and (ii) a reporter gene, wherein (i) and (ii) are under the control of the same promoter.

10. A method of modifying gene expression in a mammalian cell, comprising
delivering a mirtron or gene capable of expressing a mirtron as defined in claim 1 to a mammalian cell in vitro.


SEQUENCE LISTING (152 sequences)

SEQ ID NO: 1 - DNA, 86 bp, Unknown
  Mammalian mirtron sequence
gtagaggagatggcgcaggggacacgggcaaagacttgggggttcctgggaccctcagacgtgtgtcctcttctccctcctcccag

SEQ ID NO: 2 - DNA, 85 bp, Unknown
  Mammalian mirtron sequence
gtgaggactcgggaggtggagggtggtgccgccggggccgggcgctgtttcagctcgcttctccccccacctcctctctcctcag

SEQ ID NO: 3 - DNA, 90 bp, Unknown
  Mammalian mirtron sequence
gtgggtacggcccagtgggggggagagggacacgccctgggctctgcccagggtgcagccggactgactgagcccctgtgccgcccccag

SEQ ID NO: 4 - DNA, 75 bp, Unknown
  Mammalian mirtron sequence
gtgagggcatgcaggcctggatggggcagctgggatggtccaaaagggtggcctcaccagccctgtgttccctag

SEQ ID NO: 5 - DNA, 88 bp, Unknown
  Mammalian mirtron sequence
gtggggccaggcggtggtgggcactgctggggtgggcacagcagccatgcagagcgggcatttgaccccgtgccacccttttccccag

SEQ ID NO: 6 - DNA, 73 bp, Unknown
  Mammalian mirtron sequence
gtgggcgggggcaggtgtgtggtgggtggtggcctgcggtgagcagggccctcacacctgcctcgccccccag

SEQ ID NO: 7 - DNA, 69 bp, Unknown
  Mammalian mirtron sequence
gtgggtagggtttgggggagagcgtgggctggggttcagggacaccctctcaccactgccctcccacag

SEQ ID NO: 8 - DNA, 73 bp, Unknown
  Mammalian mirtron sequence
gtgggtgggggcatctcggaggaggtggggggtgtggcgcccagcggatgactccgagcggctcctttcccag

SEQ ID NO: 9 - DNA, 92 bp, Unknown
  Mammalian mirtron sequence
gtcagtgtctgggcggacagctgcaggaaagggaagaccaaggcttgctgtctgtccagtctgccaccctaccctgtctgttcttgccacag

SEQ ID NO: 10 - DNA, 73 bp, Unknown
  Mammalian mirtron sequence
gtggggtggcggcgacatggcgggggcggcgggccctgcggaggctgtgcgcctgaccccgaccaccccgcag

SEQ ID NO: 11 - DNA, 82 bp, Unknown
  Mammalian mirtron sequence
gtgagtgggaggccagggcacggcagggggagctgcagggctatgggaggggccccagcgtctgagccctgtcctcccgcag

SEQ ID NO: 12 - DNA, 84 bp, Unknown
  Mammalian mirtron sequence
gtgagtgtggggtggctggggggggggggggggggccggggacggcttgggcctgcctagtcggcctgaccacccaccccacag

SEQ ID NO: 13 - DNA, 74 bp, Unknown
  Mammalian mirtron sequence
gtgggcctgggtcggtggggacggggcggctgggcgtgccctgcggccgctgctctaaccgcaccgtcccccag

SEQ ID NO: 14 - DNA, 102 bp, Unknown
  Mammalian mirtron sequence
gtgagtgacaggggaaatggggatggactggaagtgggcagcatggagctgaccttcatcatggcttggccaacataatgcctcttccccttgtctctccag

SEQ ID NO: 15 - DNA, 102 bp, Unknown
  Mammalian mirtron sequence
gtgggagggcccaggcgcgggcaggggtgggggtggcagagcgctgtcccgggggcggggccgaagcgcggcgaccgtaactccttctgctccgtcccccag

SEQ ID NO: 16 - DNA, 83 bp, Unknown
  Mammalian mirtron sequence
gtgagtgggagccccagtgtgtggttggggccatggcgggtgggcagcccagcctctgagccttcctcgtctgtctgccccag

SEQ ID NO: 17 - DNA, 105 bp, Unknown
  Mammalian mirtron sequence
gtgggtgggcaggtgggtgggaagccctgggacgctgcctcctctctcctggggcctctctcgggctgggggctggtctcagtttccccattctgcctggcctag

SEQ ID NO: 18 - DNA, 83 bp, Unknown
  Mammalian mirtron sequence
gtgggccagggccgcgggggggagcaagccatctagcattcctgggaaacgcttacatctcaccatgaccctgatcccactag

SEQ ID NO: 19 - DNA, 84 bp, Unknown
  Mammalian mirtron sequence
gtgagggggctggcatggcgaggaggcgccagagaagccatagtgtggggatgggctgcacactcacctctctgtgccttccag

SEQ ID NO: 20 - DNA, 103 bp, Macaca sp.
gtgagtctgggtggggtgcagggccggcgggtgtgggctgtgggcagcaggtagaggcagacagtcaccctgaaccccgtctctcccatctgcccaccgtcag

SEQ ID NO: 21 - DNA, 86 bp, Macaca sp.
gtgagcttggcggggctgctggaggagtgggttcgcccagtctgggcaccagacacggagactccagcccacctccttctccccag

SEQ ID NO: 22 - DNA, 90 bp, Homo sapiens
gtgggtgggctgggcggggggcggggcaggtgggcggaggccctggggctctgcatagcagcagccccatgccccacctccctgtcccag

SEQ ID NO: 23 - DNA, 78 bp, Homo sapiens
gtgagtggccaccatgcggggacaggggcaggggcagccctcacccacagcctctcacctgcctttgtccacccacag

SEQ ID NO: 24 - DNA, 77 bp, Pan troglodytes
gtgagggcaaggctggggggcccctgggctaagtgggagcctggctggaattcccactccaccttactctcctgcag

SEQ ID NO: 25 - DNA, 88 bp, Homo sapiens
gtgagtgtggggcgcgccgggctgtggcgggctgggggcgggcggccctgggtcccagcctcctgctgcccaccgctgcccaccgcag

SEQ ID NO: 26 - DNA, 89 bp, Macaca sp.
gtgagcagagtgggttggaggggggtgtcccaggctctttcttgtctatgggcctgacaccccgaccctgactggcctgggcctcccag

SEQ ID NO: 27 - DNA, 94 bp, Macaca sp.
gtgaggaggcaggcggggaatgcctgagccgcagggggcctgggcctggatcccagccggcccagatttattttcatctcctgcttcctgccag

SEQ ID NO: 28 - DNA, 94 bp, Mus sp.
gtagtctgggagctggcaccgcagactatccccaggggacacggggacttggctgaggcagctccactgagagctgagagccttatccccgcag

SEQ ID NO: 29 - DNA, 82 bp, Mus sp.
gtaaaggctgggcttagacgtggcctttgggtgtggaatgcacttccgtttgtaaccgccatctaaccctggcctttgacag

SEQ ID NO: 30 - DNA, 79 bp, Homo sapiens
gtgagctggggtggctgaggcgggatgggggccacctgaggctggggctggccctgctcactgctgctcctgcccacag

SEQ ID NO: 31 - DNA, 82 bp, Homo sapiens
gtgggcgaacgcccgggtggagcgggtcaggcagggccagtcacagacacagctctgtgctgactgcccgccttgcccacag

SEQ ID NO: 32 - DNA, 181 bp, Homo sapiens
gtgagcagggccagtgctggactctgctgctgggccttggtgggcaggggcagcaagagaggttcagctttggccctggcccagctctctgggcacaggggtcagctgggatgggagtctggagaacaaggggtccctgaagaccagcaaccatctcactctggcacccctgctcccccag

SEQ ID NO: 33 - DNA, 75 bp, Homo sapiens
gtagggccccgccgagggggcagggtgggggccccagggacccccctcacggcctgcggtctgggctctcggcag

SEQ ID NO: 34 - DNA, 133 bp, Homo sapiens
ccagagtgcgtgaggacacgtagaggggctcaggctgccaagggggcacggagcttgtgggagcaccaggactgggactgttcatgtgggtgtcctctgccctccctgaccccttgcctgtctctcgccacag

SEQ ID NO: 35 - DNA, 90 bp, Homo sapiens
gtaggcagaggggcagggtggtggcgggggagagggtaggggggcggggccgcagtgctcagctgtcttcccctcggccctgccccacag

SEQ ID NO: 36 - DNA, 74 bp, Homo sapiens
gtgcgtggtggctcgaggcgggggtgggggcctcgccctgcttgggccctccctgacctctccgctccgcacag

SEQ ID NO: 37 - DNA, 75 bp, Homo sapiens
gtgagggcagccggcagggccccaggtcctgcttacatgtgggcccagactccagctccctctccccacatgcag

SEQ ID NO: 38 - DNA, 87 bp, Homo sapiens
gtgaggcggggccaggagggtgtgtggcgtgggtgctgcggggccgtcagggtgcctgcgggacgctcacctggctggcccgcccag

SEQ ID NO: 39 - DNA, 80 bp, Homo sapiens
gtgagtggggtgggggtgtggggtggggggcatggagccggcgtggaaccagagccctcactcctgcccacacccctcag

SEQ ID NO: 40 - DNA, 97 bp, Homo sapiens
gtgagcggccgctgggggcaggtggcgggtgggagggagggaggtgggctttcccggtgggcgttcagaaacccccttttacctgactctctcccag

SEQ ID NO: 41 - DNA, 92 bp, Homo sapiens
gtgagccagtggaatggagaggctgtgggcagggggagatgtgaaggaaagaactaggacccattcatccactgcattcctgcttggcccag

SEQ ID NO: 42 - DNA, 104 bp, Homo sapiens
gtgaggctggggcagggatgaggtggcccaggagagtgagtccccgagggtgggccgcggaaagccagcaggctgaccaggccttcgtgtcccctccctgccag

SEQ ID NO: 43 - DNA, 77 bp, Homo sapiens
gtaggggcggggtgggagaggtggggtctgcgtgcgggccgggccggcccctgacgcgcgctcctcccctgccccag

SEQ ID NO: 44 - DNA, 73 bp, Homo sapiens
gtgggtgcggaggcggggggacagggtacccacgaggcctgaactcacacctcggcgcctcctggccccgcag

SEQ ID NO: 45 - DNA, 88 bp, Homo sapiens
gtgagcccgcagcggggcagggtacaggggcgggggcccccatggccaggccccaccccgctcctattgaccctcctgctgtcctcag

SEQ ID NO: 46 - DNA, 72 bp, Homo sapiens
gtgagggggctgccaggggtaggctacaggcctccatcacgggggacccctctgaagccaccccctccccag

SEQ ID NO: 47 - DNA, 85 bp, Homo sapiens
gtgaggggctggggttttcgtcccatggcgggcagggcccagggagctgaggctgcttctaaagccatctccccggtacccgcag

SEQ ID NO: 48 - DNA, 68 bp, Homo sapiens
gtaggtcaggcccaggtgtgggttggggtccggctgcagcgcccgctaacccacctgctgctgtccag

SEQ ID NO: 49 - DNA, 92 bp, Pan troglodytes
gtgagtgggcagggacaggagcctgcgggctggggttggggggcacagtgggtgtgcccgcttggcctgtgaccacctgcactgtgccccag

SEQ ID NO: 50 - DNA, 85 bp, Pan troglodytes
gtgagtgggggcttcaggcttcagggcgggggggaggggaacgccggaggcccctccgcggccctcacctaccctcccccgccag

SEQ ID NO: 51 - DNA, 83 bp, Macaca sp.
gtgagcagggatggcgggcattgggggagccgggagcgggggacagccggggtccctctcacgcccgcctcgctgcccggcag

SEQ ID NO: 52 - DNA, 85 bp, Macaca sp.
gtgagcacgcgggatttccaggggacgggcaggacgctgcagtctgactggcgcaggggcctgggaatccgcctgtctcccacag

SEQ ID NO: 53 - DNA, 113 bp, Macaca sp.
gtacggaaggccagggtggtggcgggaatccattccggtgtgttctacacattgcttagagattggatggggtgcatcagggtggtttctgattacttttcttcttccctcag

SEQ ID NO: 54 - DNA, 70 bp, Macaca sp.
gtgagctgggggatgtgaggggttggactgttccagttctctgtctaccctctctgcctcctgcctgcag

SEQ ID NO: 55 - DNA, 93 bp, Macaca sp.
gtgaggcgggcagacggcatgggagggcaggcgggctgggtttctgactggccctggctgaccgtgcttccctcttccctctgccccgcccag

SEQ ID NO: 56 - DNA, 81 bp, Macaca sp.
gtgaggccgacgcgaggcggctgcgagaccccgtgcggccttgcgggcgcgccctcaccctctgccgtccctgtctcacag

SEQ ID NO: 57 - DNA, 74 bp, Macaca sp.
gtgagtgtggggtggctggggtggggcagggatggcttgggcctgcccagtcggcctgaccacccaccccacag

SEQ ID NO: 58 - DNA, 92 bp, Macaca sp.
gtgagcagggcgatcgggggggctggcgtgctggggtggggcacagggggtgcctcagccgcctctgagctcccacaacccgcccctgccag

SEQ ID NO: 59 - DNA, 166 bp, Macaca sp.
gtgagtaggggcgggaggaggggacaccctgagctcagtccagccaggagaggcattggggtgtccctgccatctggccgtggaggggcaaggcaggaggtatgtgccatgggtgggggcagttgagctgaggtaggaggtgaccctgcctgccacctgcctttag

SEQ ID NO: 60 - DNA, 67 bp, Macaca sp.
gtaggtgcggcggtgcggctgtgggagggctgccggtggcgctccctctgagcctgccgcctcgcag

SEQ ID NO: 61 - DNA, 93 bp, Macaca sp.
gtgggagcaggcagggctgggcaggccaggggtctgggaggcaggggcccgggcagccctgaacccaacccctggcttcctctcccatcccag

SEQ ID NO: 62 - DNA, 98 bp, Macaca sp.
gtgagccagagtggtggagcagggaggcgggggttcagccagggcttgctgggccagccagagcgcccccgtgatgccctgcggctctgttctcacag

SEQ ID NO: 63 - DNA, 96 bp, Mus sp.
gtgagagcggggggagtggcaagtgagacggatgggcggggctggctcagactgctagccccgcccagctctctaactcttcccttgtgccctcag

SEQ ID NO: 64 - DNA, 74 bp, Mus sp.
gtgaggctcagtatggggtgggggtgtcgtcgcctgcccgactgaccacccactcaccctggactgactctcag

SEQ ID NO: 65 - DNA, 22 bp, Artificial
  forward primer
agatccgctagcgctaccggtc

SEQ ID NO: 66 - DNA, 37 bp, Artificial
  reverse primer
gtctactcgagaagactacttgaagaagatggtgcgc

SEQ ID NO: 67 - DNA, 37 bp, Artificial
  forward primer
gtcttctcgagaagacttgacgacggcaactacaaga

SEQ ID NO: 68 - DNA, 29 bp, Artificial
  reverse primer
atcgaagcttacttgtacagctcgtccat

SEQ ID NO: 69 - DNA, 91 bp, Homo sapiens
gtatgtagtagaattctgtcaatctttttggttgtctcagattttaattttattagcagcatgagattgactctttcataatctacttaag

SEQ ID NO: 70 - DNA, 86 bp, Homo sapiens; identical to SEQ ID NO: 1

SEQ ID NO: 71 - DNA, 86 bp, Artificial
  miR-877 with 5′ splice site mutation to prevent splicing
agtggggagatggcgcaggggacacgggcaaagacttgggggttcctgggaccctcagacgtgtgtcctcttctccctcctcccag

SEQ ID NO: 72 - DNA, 86 bp, Artificial
  miR-877 with branch point mutation to prevent splicing
gtagaggagatggcgcaggggacacgggcaaagacttgggggttcctgggaccctccggcgtgtgtcctcttctccctcctcccag

SEQ ID NO: 73 - DNA, 75 bp, Homo sapiens; identical to SEQ ID NO: 4

SEQ ID NO: 74 - DNA, 75 bp, Artificial
  miR-1226 with 5′ splice site mutation to prevent splicing
acgagggcatgcaggcctggatggggcagctgggatggtccaaaagggtggcctcaccagccctgtgttccctag

SEQ ID NO: 75 - DNA, 75 bp, Artificial
  miR- 1226 with 5′ and 3′ splice site mutations to prevent splicing
acgagggcatgcaggcctggatggggcagctgggatggtccaaaagggtggcctcaccagccctgtgttccctga

SEQ ID NO: 76 - DNA, 75 bp, Artificial
  miR- 1226 with branch point mutation to prevent splicing
gtgagggcatgcaggcctggatggggcagctgggatggtccgggggggtggcctcaccagccctgtgttccctag

SEQ ID NO: 77 - DNA, 85 bp, Mus sp.
gtgaggactggggaggtggagggtagcatcattagagccagagctctgtctcagctccctctccccccacctcttctctcctcag

SEQ ID NO: 78 - DNA, 85 bp, Artificial
  miR-1224 sequence with 5′  and 3′ splice site mutations to prevent splicing
gcgaggactggggaggtggagggtagcatcattagagccagagctctgtctcagctccctctccccccacctcttctctcctcgg

SEQ ID NO: 79 - DNA, 69 bp, Artificial
  mirt1.1 targeting cycB
gtgggtttttggaacagtctttcctttgttctggtctgacccatgggaaagactgttccaaaaattcag

SEQ ID NO: 80 - DNA, 65 bp, Artificial
  mirt3.1 targeting cycB
gtgggtcaaaatacaccttgacggtgttctggtctgacccatcgtcaaggtgtattttgacccag

SEQ ID NO: 81 - DNA, 65 bp, Artificial
  mirt 3.2 targeting cycB
gtgggaatttgctgtttttgtagctgttctggtctgacccagctacaaaaacagcaaattcccag

SEQ ID NO: 82 - DNA, 63 bp, Artificial
  mirt 5.1 targeting cycB
gtggggaagaactgggagcccgtgttctggtctgacccatggctcccagttcttctccaccag

SEQ ID NO: 83 - DNA, 62 bp, Artificial
  mirt 5.2 targeting cycB
gtgggtcagtttgaagttctctgttctggtctgacccagggaacttcaaactgatccaccag

SEQ ID NO: 84 - DNA, 86 bp, Artificial
  mirt11 targeting cycB
gtaggtcaaaatacaccttggacacgggcaaagacttgggggttcctgggaccctcagacgtgtgtccaagcttattttcctccag

SEQ ID NO: 85 - DNA, 86 bp, Artificial
  mirt12 targeting cycB
gtggatcatgaagtccttgagacacgggcaaagacttgggggttcctgggaccctcagacgtgtgtctcaagtccttcttccccag

SEQ ID NO: 86 - DNA, 86 bp, Artificial
  mirt13 targeting cycB
gtgatgaagaactgggagccgacacgggcaaagacttgggggttcctgggaccctcagacgtgtgtcggctcccttcttctcccag

SEQ ID NO: 87 - DNA, 88 bp, Artificial
  mirt14 targeting cycB
gtcagtttgaagttctcatcgacacgggcaaagacttgggggttcctgggaccctcagacgtgtgtcgatgagaacttcccactccag

SEQ ID NO: 88 - DNA, 86 bp, Artificial
  mirtFL1 targeting Luc
gtgagatgtcacgaatgtgtgacacgggcaaagacttgggggttcctgggaccctcagacgtgtgtcacacctccgacatctccag

SEQ ID NO: 89 - DNA, 86 bp, Artificial
  mirtFL2 targeting Luc
gtgagaaatgcccatgctgtgacacgggcaaagacttgggggttcctgggaccctcagacgtgtgtcacagtacaaatttctccag

SEQ ID NO: 90 - DNA, 75 bp, Artificial
  mirt21 targeting cycB
gtgaggacagactgtcccaagaaaggcagctgggatggtccaaaagggtggcctttttggaacagtctttcctag

SEQ ID NO: 91 - DNA, 95 bp, Artificial
  mirt21B targeting cycB
gtgaggacagactgtcccaagaaaggacacgggcaaagacttgggggttcctgggaccctcagacgtgtgtcctttttggaacagtctttcctag

SEQ ID NO: 92 - DNA, 75 bp, Artificial
  mirt21C targeting cycB
gtgaggacagactgtcccaagaaaggcagctgggatggtccagacgggtggcctttttggaacagtctttcctag

SEQ ID NO: 93 - DNA, 75 bp, Artificial
  mirt21OBP targeting cycB
gtgaggacagactgtcccaagaaaggcagctgggatggtcctgacgggtggcctttttggaacagtctttcctag

SEQ ID NO: 94 - DNA, 85 bp, Artificial
  pMirt A-syn 1
gtggagcctacatagagaacaggtagcatcattagagccagagctctgtctcagctccttctccccttctctatgttcgctccag

SEQ ID NO: 95 - RNA, 75 nt, Artificial
  pMirt A-syn 2
gugacagaggcugaggagacacaaggcaggagggaugguccagacguccugccuuggucuucucagccacuguag

SEQ ID NO: 96 - RNA, 85 nt, Artificial
  pMirt A-syn 3
gugcgugagugagaggaccgggguagcaucauuagagccagagcucugucucagcucccuccccuuggucuucucagccauguag

SEQ ID NO: 97 - RNA, 75 nt, Artificial
  pMirt A-syn 4
gugggcaaaagcagccggaagagaggcaggagggaugguccagacguccugccucuuuccugcugcuucugccag

SEQ ID NO: 98 - RNA, 85 nt, Artificial
  pMirt A-syn 5
gugcggccuuagcccggaggagguagcaucauuagagccagagcucugucucagcucccuccccucuucugggcuacugcuguag

SEQ ID NO: 99 - RNA, 75 nt, Artificial
  pMirt A-syn 6
gugggcaccaguagcgcagaaagaggcaggagggaugguccagacguccugccucuucugggcuacugcugucag

SEQ ID NO: 100 - RNA, 39 nt, Artificial
  example of acceptable branch point
cgggcaaagacuuggggguuccugggacccucagacgug

SEQ ID NO: 101 - RNA, 21 nt, Artificial
  example of acceptable branch point sequence
gcugggaugguccugacgggu

SEQ ID NO: 102 - RNA, 86 nt, Homo sapiens
guagaggagauggcgcaggggacacgggcaaagacuuggggguuccugggacccucagacguguguccucuucucccuccucccag

SEQ ID NO: 103 - RNA, 75 nt, Homo sapiens
gugagggcaugcaggccuggauggggcagcugggaugguccaaaaggguggccucaccagcccuguguucccuag

SEQ ID NO: 104 - RNA, 85 nt, Mus sp.
gugaggacuggggagguggaggguagcaucauuagagccagagcucugucucagcucccucuccccccaccucuucucuccucag

SEQ ID NO: 105 - RNA, 69 nt, Artificial
  mirt1.1 targeting cycB
guggguuuuuggaacagucuuuccuuuguucuggucugacccaugggaaagacuguuccaaaaauucag

SEQ ID NO: 106 - RNA, 65 nt, Artificial
  mirt3.1 targeting cycB
gugggucaaaauacaccuugacgguguucuggucugacccaucgucaagguguauuuugacccag

SEQ ID NO: 107 - RNA, 65 nt, Artificial
  mirt3.2 targeting cycB
gugggaauuugcuguuuuuguagcuguucuggucugacccagcuacaaaaacagcaaauucccag

SEQ ID NO: 108 - RNA, 63 nt, Artificial
  mirt5.1 targeting cycB
guggggaagaacugggagcccguguucuggucugacccauggcucccaguucuucuccaccag

SEQ ID NO: 109 - RNA, 62 nt, Artificial
  mirt5.2 targeting cycB
gugggucaguuugaaguucucuguucuggucugacccagggaacuucaaacugauccaccag

SEQ ID NO: 110 - RNA, 86 nt, Artificial
  mirt11 targeting cycB
guaggucaaaauacaccuuggacacgggcaaagacuuggggguuccugggacccucagacguguguccaagcuuauuuuccuccag

SEQ ID NO: 111 - RNA, 86 nt, Artificial
  mirt12 targeting cycB
guggaucaugaaguccuugagacacgggcaaagacuuggggguuccugggacccucagacgugugucucaaguccuucuuccccag

SEQ ID NO: 112 - RNA, 86 nt, Artificial
  mirt13 targeting cycB
gugaugaagaacugggagccgacacgggcaaagacuuggggguuccugggacccucagacgugugucggcucccuucuucucccag

SEQ ID NO: 113 - RNA, 88 nt, Artificial
  mirt14 targeting cycB
gucaguuugaaguucucaucgacacgggcaaagacuuggggguuccugggacccucagacgugugucgaugagaacuucccacuccag

SEQ ID NO: 114 - RNA, 86 nt, Artificial
  mirtFL1 targeting Luc
gugagaugucacgaaugugugacacgggcaaagacuuggggguuccugggacccucagacgugugucacaccuccgacaucuccag

SEQ ID NO: 115 - RNA, 86 nt, Artificial
  mirtFL2 targeting Luc
gugagaaaugcccaugcugugacacgggcaaagacuuggggguuccugggacccucagacgugugucacaguacaaauuucuccag

SEQ ID NO: 116 - RNA, 75 nt, Artificial
  mirt21 targeting cycB
gugaggacagacugucccaagaaaggcagcugggaugguccaaaaggguggccuuuuuggaacagucuuuccuag

SEQ ID NO: 117 - RNA, 95 nt, Artificial
  mirt21B targeting cycB
gugaggacagacugucccaagaaaggacacgggcaaagacuuggggguuccugggacccucagacguguguccuuuuuggaacagucuuuccuag

SEQ ID NO: 118 - RNA, 75 nt, Artificial
  mirt21C targeting cycB
gugaggacagacugucccaagaaaggcagcugggaugguccagacggguggccuuuuuggaacagucuuuccuag

SEQ ID NO: 119 - RNA, 75 nt, Artificial
  mirt21OBP targeting cycB
gugaggacagacugucccaagaaaggcagcugggaugguccugacggguggccuuuuuggaacagucuuuccuag

SEQ ID NO: 120 - RNA, 85 nt, Artificial
  ASyn 1
guggagccuacauagagaacagguagcaucauuagagccagagcucugucucagcuccuucuccccuucucuauguucgcuccag

SEQ ID NO: 121 - DNA, 75 bp, Artificial
  ASyn 2
gugacagaggctgaggagacacaaggcaggagggaugguccagacguccugccttggtcttctcagccactgtag

SEQ ID NO: 122 - DNA, 85 bp, Artificial
  ASyn 3
gugcgtgagtgagaggaccgggguagcaucauuagagccagagcucugucucagcucccuccccttggtcttctcagccatgtag

SEQ ID NO: 123 - DNA, 85 bp, Artificial
  ASyn 5
gugcggccuuagcccggaggagguagcaucauuagagccagagcucugucucagcucccucccctcttctgggctactgctgtag

SEQ ID NO: 124 - DNA, 75 bp, Artificial
  ASyn 6
gugggcaccagtagcgcagaaagaggcaggagggaugguccagacgtcctgcctcttctgggctactgctgtcag

SEQ ID NO: 125 - RNA, 86 nt, Artificial
  Unspliceable miR-877 mutant
auagaggagauggcgcaggggacacgggcaaagacuuggggguuccugggacccucagacguguguccucuucucccuccucccag

SEQ ID NO: 126 - RNA, 86 nt, Artificial
  double unspliceable miR-877 mutant
gcagaggagauggcgcaggggacacgggcaaagacuuggggguuccugggacccucagacguguguccucuucucccuccucccga

SEQ ID NO: 127 - RNA, 86 nt, Artificial
  scrambled miR-877 mutant
guagaggggccggugugggggacacgggcaaagacuuggggguuccugggacccucagacguguguccucuucucccuccucccag

SEQ ID NO: 128 - RNA, 86 nt, Artificial
  Branch point miR-877 mutant
guagaggagauggcgcaggggacacgggcaaagacuuggggguuccugggacccuccggcguguguccucuucucccuccucccag

SEQ ID NO: 129 - RNA, 21 nt, Artificial
  sense siRNA
ggaaagacuguuccaaaaauu

SEQ ID NO: 130 - RNA, 21 nt, Artificial
  antisense siRNA
uuuuuggaacagucuuuccuu

SEQ ID NO: 131 - DNA, 21 bp, Artificial
  target mirtron sequence
ggacagcgtgcccccctttac

SEQ ID NO: 132 - DNA, 21 bp, Artificial
  target mirtron sequence
gcccaccgcctgccagttcac

SEQ ID NO: 133 - DNA, 21 bp, Artificial
  target mirtron sequence
tggagccctcggtgtccccac

SEQ ID NO: 134 - DNA, 21 bp, Artificial
  target mirtron sequence
gaccttctcctgggcgtccac

SEQ ID NO: 135 - DNA, 21 bp, Artificial
  target mirtron sequence
gcccttctacgcggattccac

SEQ ID NO: 136 - DNA, 21 bp, Artificial
  target mirtron sequence
ccttttgtgggctactcctac

SEQ ID NO: 137 - DNA, 21 bp, Artificial
  target mirtron sequence
ggagagggacgtgttggtgaa

SEQ ID NO: 138 - DNA, 21 bp, Artificial
  target mirtron sequence
gcttaaggaggtccgactgca

SEQ ID NO: 139 - DNA, 21 bp, Artificial
  target mirtron sequence
ggaggtccgactgcagaggga

SEQ ID NO: 140 - DNA, 21 bp, Artificial
  target mirtron sequence
cactacaaggagcacctctct

SEQ ID NO: 141 - DNA, 21 bp, Artificial
  target mirtron sequence
ctacaaggagcacctctctct

SEQ ID NO: 142 - DNA, 21 bp, Artificial
  target mirtron sequence
cctgaggaggctcgagacttc

SEQ ID NO: 143 - DNA, 21 bp, Artificial
  target mirtron sequence
gaggaggctcgagacttcatt

SEQ ID NO: 144 - DNA, 21 bp, Artificial
  target mirtron sequence
caacttcgacttggtggagga

SEQ ID NO: 145 - DNA, 21 bp, Artificial
  target mirtron sequence
cggggagacactgtcggacat

SEQ ID NO: 146 - DNA, 84 bp, Artificial
  synthetic mirtron
gtgaaggggggcacgctgtccggtagcatcattagagccagagctctgtctcagctcctgactttcctgtctgccccttttcag

SEQ ID NO: 147 - DNA, 83 bp, Artificial
  synthetic mirtron
gtggagagggacgtgttgctgggaggcaaagacttgggggttcctgggaccctcagacgtttcaccaacacgtctttctccag

SEQ ID NO: 148 - DNA, 21 bp, Artificial
  DMPK Mirt5 target sequence
catcctcatcgggtgttttcc

SEQ ID NO: 149 - DNA, 79 bp, Artificial
  DMPK Mirt5
gtaggagtagcccacaaaaggaaagcatcattagagccagagctctgtctcagctttccttttgtgttctactttttag

SEQ ID NO: 150 - protein, 7 aa, Artificial
  Translation of mirtron target sequence
PFVGYSY

SEQ ID NO: 151 - DNA, 21 bp, Artificial
  codon replaced target of Mirt5
ccattcgtcgggtatagctat

SEQ ID NO: 152 - DNA, 20 bp, Artificial
  miR-877 target
ccctgcgccatctcctctac